SEQUENCE LISTING (2 sequences)

SEQ ID NO: 1 - protein, 10 aa, Artificial Sequence
  A synthetic sequence
SAVLQSGFRK

SEQ ID NO: 2 - protein, 9 aa, Artificial Sequence
  A synthetic sequence
AVLQSGFRK

(19) United States
(12) Patent Application Publication
(10) Pub. No.: US 2023/0301988 A1
(43) Pub. Date: Sep. 28, 2023

(54) INHIBITORS OF SARS COV-2 INFECTION AND USES THEREOF

(71) Applicants: The Texas A&M University System, College Station, TX (US); The Board of Regents of the University of Texas System, Austin, TX (US)

(72) Inventors: Thomas D. Meek, College Station, TX (US); Chien-Te Tseng, Galveston, TX (US); Aleksandra Drelich, Galveston, TX (US); Drake M. Mellott, College Station, TX (US)

(21) Appl. No.: 18/023,661

(22) PCT Filed: Aug. 26, 2021
(86) PCT No.: PCT/US2021/047782

§ 371 Date: Feb. 27, 2023

Related U.S. Application Data
(60) Provisional application No. 63/088,154, filed on Oct. 6, 2020.
(60) Provisional application No. 63/071,163, filed on Aug. 27, 2020.

(51) Int. Cl. A61K 31/495; A61P 31/14; G01N 33/569

(52) U.S. Cl. CPC A61K 31/495; A61P 31/14; G01N 33/56983; G01N 2333/165

(57) ABSTRACT

Provided herein are pharmaceutical compositions including compounds having Formula I (I) in an effective amount to inhibit non-viral cysteine protease (e.g., mammalian cysteine protease, such as human cathepsin L), as well as methods of using thereof.

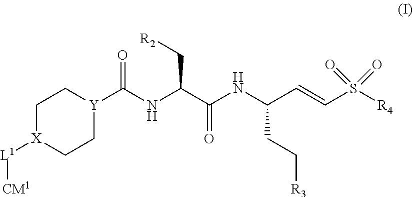

### CROSS-REFERENCE TO RELATED APPLICATIONS

This application claims the benefit of U.S. Provisional Appl. Ser. No. 63/088,154, filed Oct. 6, 2020 and 63/071,163, filed Aug. 27, 2020, the entirety of both of which is incorporated by reference as if fully set forth herein.

### BACKGROUND

The outbreak of COVID-19 in 2019 has precipitated a global health crisis of a scale unseen since the 1918/1919 influenza. As of August 2020, 20 million people are infected worldwide, including more than 5 million in the US. At this writing, no approved vaccines or therapies exist for the treatment of COVID-19, which may significantly prolong the current worldwide health and economic crisis that this virus has wrought. Accordingly, the urgency for the identification of drugs or clinical-stage compounds that limit the pathology caused by SARS-CoV-2 for the management of COVID-19 cannot be overstated.

The severe acute respiratory syndrome (SARS) and Middle East respiratory syndrome (MERS) outbreaks of 2003 and 2012, respectively, caused by betacoronaviruses that are highly related to SARS CoV-2, provided important precedents for the means to block infection from the highly homologous SARS CoV-2. Entry into permissive mammalian cells by CoV-2, like its predecessor, requires the binding of a trimeric coronaviral envelope glycoprotein, called the Spike protein. The Spike protein of SARS-CoV-2, has 76% amino acid identity with that of SARS-CoV-1, and in kind, effects the binding and fusion of the viral envelope with mammalian cell membranes. This binding and fusion occurs between the coronaviral Spike protein and an extracellular region of the angiotensin converting enzyme-2 (ACE2) found on the surface of virally permissive cells. This viral-cell binding is the first step in host-cell penetrance. Based on data of the mechanism of viral entry of SARS CoV-1, several proteolytic events occur at the S2/S1 domains of the Spike protein, catalyzed by the serine proteases furin and transmembrane protease serine-2 (TMPRSS-2) of the host cell, to prepare the virus for cellular penetrance. Following this, the action of one or more host-cell cysteine proteases on the now-modified Spike protein enables the deposition of the coronaviral proteins and RNA into the host cells. This step is essential to the establishment of infection of the host cell. These findings have elicited studies to explore the role of (generic) inhibitors of serine and cysteine proteases in inhibiting coronaviral entry.

Cysteine proteases comprise a large family of proteases that catalyze cleavages of other proteins, including other proteases, that leads to their activation or inactivation. Members of the papain sub-family of cysteine proteases (known as Clan CA) have similar active sites structures, containing a cysteine residue and a closely-associated, conserved histidine residue, suggesting that cysteine proteases of the papain sub-family have similar chemical mechanisms, and thereby, may be inhibited or covalently inactivated by related compounds. Aberrant activities of the papain-like cysteine proteases (names in parentheses below) result in a broad panel of diseases, many of which are of unmet or under-met medical needs, including malaria (falcipains-2 and -3), Chagas disease (cruzain/cruzipain), African sleeping sickness (TbCatB, TbCatL, rhodesain), some cancers (caspases and cathepsin L), osteoporosis (cathepsin K), and respiratory ailments including COPD and asthma (cathepsins C and S). Recently, the action of cysteine proteases, both those that are virally-encoded by SARS-CoV-2, as well as those found on or near the surface of permissive host cells, has been shown to be involved in the infection of primate and human cells by SARS-CoV-2. Several existing inhibitors or irreversible inactivators of cysteine proteases have been shown to inhibit the infection of mammalian cells by SARS CoV-2.

### BRIEF DESCRIPTION OF THE FIGURES

The drawings illustrate generally, by way of example, but not by way of limitation, various embodiments discussed herein.

FIG. 1 is a synthetic scheme for K11777.

FIG. 2 is a synthetic scheme for Propargyl-K11777.

FIG. 3 is a table showing the effects of K777 vs. cruzain and human cysteine proteases. Values of ICs& indicate the concentrations of K777 that reduce enzymatic activity by 50%.

FIG. 4 is a table showing the effects of K777 in Vero E6 and A549/ACE2 cells, infected with SARS CoV-2. Measurement of the cytopathic effect (CPE) indicates the extent to which SAR-CoV-2 has killed the host cells, and values of EC₅₀ indicate the concentrations of K777 that reduce the CPE by 50%.

FIG. 5A is a graph showing the initial velocity data (relative fluorescent units/sec) obtained with 5-160 micromolar Abz-Ser-Ala-Val-Leu-Gln*Ser-Gly-Phe-Arg-Lys-(DNP)-NH2 and 50 nM 3CLpro.

FIG. 5B is a graph showing the time course data of 3CLpro-catalyzed cleavage of Ala-Val-Leu-Gin*Ser-Gly-Phe-Arg-Lys-(DNP)-NH2 in the presence of 0 (red), 0.2 mM (green), 1.0 mM (blue) and 5.0 micromolar (magenta) concentrations of K777.

FIG. 6A-6E show that K777 alkyne specifically targets a non-viral protein in both SARS-CoV-2 infected and uninfected Vero E6 cells. FIG. 6A is an image of an in-gel fluorescence of Cy7 azide labeled proteins in both SARS-CoV-2 infected and uninfected cells at 1 μM K777 alkyne is blocked by pre-treatment with 1 μM K777. FIG. 6B is a graph showing a densitometry analysis of FIG. 6A, and a replicate in gel fluorescence experiment. FIG. 6C is an image of a western blot of total p-actin levels in SARS-CoV-2 infected and non-infected cells from 20 μg of total lysate in duplicate. FIG. GD is a graph showing a densitometry analysis of the western blots in FIG. 6C. FIG. 6E is a graph showing the comparison of the in signal intensity of the 25-kDa enriched band in the presence of K777 alkyne in both SARS-CoV-2 infected and uninfected cells versus the p-actin signal in SARS-CoV-2 infected and unifected cells.

FIG. 7A-7B shows that cathepsins B and L are strongly enriched upon K777 alkyne treatment. FIG. 7A is a schematic of enrichment strategy employed to prepare samples for proteomic processing. FIG. 7B is a heat map of proteomic data clustered from highest to lowest intensity. Intensity reports on the summation of the peptides intensities within a particular protein group.

FIG. 8A-8D shows that CTSL and CTSB catalyze differential processing of the SARS-CoV-2 S protein which is inhibited by K777. FIG. 8A. Processing of SARS1-S protein and SARS2 S protein by CTSB (250 nM) and CTSL (25 nM) FIG. 8B. Concentration-dependent cleavage of the SARS-CoV-2 spike protein by CTSL yields a ˜120 kDa polypeptide, a ˜40 kDa polypeptide, and smaller protein fragments. FIG. 8C. Western blotting of the FLAG epitope of the C-terminus of the SARS-CoV-2-S protein demonstrates that CTSL (25 nM) processing produces a signal that localized at ˜120 kDa, suggesting that cleavage by CTSL in the S1 domain. FIG. 8D. CTSL-catalyzed (25 nM) proteolysis of the SARS-2 S spike protein yields a peptide of ˜120 kDa and is inhibited by K777, CTSB (250 nM) does not catalyze proteolysis of the spike protein.

### SUMMARY

It would therefore be advantageous to have an inhibitor of cysteine proteases that has low nanomolar potency for its enzyme target and is a reversible inactivator of the enzyme.

Described herein are pharmaceutical compositions comprising a compound having Formula I or pharmaceutically acceptable salts or derivatives thereof, and one or more pharmaceutically acceptable carriers. In some embodiments, the compound is present in an effective amount to inhibit a non-viral cysteine protease.

In some embodiments, R₁ is an heteroaryl such as a N-methyl-piperazinyl or N-propynyl-piperazinyl. In some embodiments, R₂ and R₄ are phenyl. In some embodiments, Ra is benzyl. In some embodiments, the non-viral cysteine protease is a mammalian cysteine protease such as cathepsin L. In some embodiments, the non-viral cysteine protease is selected from cruzain, cruzipain, cathepsin L, cathepsin B, cathepsin K, cathepsin S, cathepsin V, or other cysteine proteases.

The present disclosure also provides methods for treating, inhibiting, decreasing, reducing, ameliorating and/or preventing coronavirus infections in a subject in need thereof, the method comprising administering a therapeutically effective amount of the compound, or a pharmaceutically acceptable salt or derivative thereof. In another aspect, the present disclosure provides methods for treating, inhibiting, decreasing, reducing, ameliorating and/or preventing the disease and/or symptoms associated with a coronavirus infection in a subject in need thereof, comprising administering a therapeutically effective amount of the compound, or a pharmaceutically acceptable salt or derivative thereof. As used herein, the term “coronavirus” generally refers to coronaviruses such as SARS-related coronaviruses (SARSr-CoVs) including the highly pathogenic zoonotic pathogens SARS-CoV (also known as SARS-CoV-1), MERS-CoV, and SARS-CoV-2, all belonging to the b-coronavirus genus. The term “coronavirus” also includes the four low-pathogenicity coronaviruses that are endemic in humans, namely, HCoV-OC43, HCoVHKU1, HCoV-NL63, and HCoV-229. The term “coronavirus” also includes any variants of coronavirus such as the so-called delta variant, which has become the most prevalent “form” of the novel coronavirus, also known as SARS-CoV-2. The disclosure therefore also provides methods for treating, inhibiting, decreasing, reducing, ameliorating and/or preventing coronavirus infections caused by SARS-CoV-2 in a subject in need thereof, the method comprising administering a therapeutically effective amount of the compound, or a pharmaceutically acceptable salt or derivative thereof. In another aspect, the present disclosure provides methods for treating, inhibiting, decreasing, reducing, ameliorating and/or preventing the disease and/or symptoms associated with a coronavirus infection caused by SARS-CoV-2 in a subject in need thereof, comprising administering a therapeutically effective amount of the compound, or a pharmaceutically acceptable salt or derivative thereof.

The disclosure also provides methods of treating a coronavirus disease, the methods comprising administering a compound having Formula I or pharmaceutically acceptable salts or derivatives thereof or a composition comprising a compound having Formula I or pharmaceutically acceptable salts or derivatives thereof, and one or more pharmaceutically acceptable carriers to a subject in need thereof. The coronavirus disease can be COVID-19.

The disclosure also relates to methods of preventing or delaying the onset or progression of a coronavirus disease, the methods comprising administering a compound having Formula I or pharmaceutically acceptable salts or derivatives thereof or a composition comprising a compound having Formula I or pharmaceutically acceptable salts or derivatives thereof, and one or more pharmaceutically acceptable carriers to a subject in need thereof. The coronavirus disease can be COVID-19.

Described are also methods for identification of a protein target of the compound having Formula I in mammalian cells or tissues infected or not-infected with coronaviruses. In some embodiments, the protein target or targets is cruzain, cruzipain, cathepsin B, cathepsin L, cathepsin S, or other cysteine proteases.

The details of one or more embodiments of the disclosure are set forth in the accompanying drawings and the description below. Other features, objects, and advantages of the disclosure will be apparent from the description and drawings, and from the claims.

### DESCRIPTION

The following description of the disclosure is provided as an enable teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

The most successful inhibitors of the papain sub-family of cysteine proteases are peptide analogues that form covalent bonds, both reversible and irreversible, with the active-site cysteines of these enzyme targets. The types of substituents that form these covalent bonds include but are not limited to epoxides, nitriles, ketones, aldehydes, propenamides and vinyl sulfones. These electrophilic “warheads” have been installed in peptidomimetic or “organic” scaffolds, and have led to clinical-stage compounds for the treatment of malaria, Chagas disease, cystic fibrosis, and osteoporosis. Vinyl-sulfone moieties as the warheads of this class of irreversible covalent inactivators can be found in compounds having formula I.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs.

### General Definitions

To facilitate understanding of the disclosure set forth herein, a number of terms are defined below. Unless defined otherwise, all technical and scientific terms used herein have the same meanings as commonly understood by one of skill in the art to which the disclosed invention belongs. Publications cited herein and the materials for which they are cited are specifically incorporated by reference.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein. It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if a composition is described herein as including a component of type A, a component of type B, a component of type C, or any combination thereof, it is understood that this phrase describes all of the various individual and collective combinations and permutations of these components. For example, in some embodiments, the composition described by this phrase could include only a component of type A. In some embodiments, the composition described by this phrase could include only a component of type B. In some embodiments, the composition described by this phrase could include only a component of type C. In some embodiments, the composition described by this phrase could include a component of type A and a component of type B. In some embodiments, the composition described by this phrase could include a component of type A and a component of type C. In some embodiments, the composition described by this phrase could include a component of type B and a component of type C. In some embodiments, the composition described by this phrase could include a component of type A, a component of type B, and a component of type C. In some embodiments, the composition described by this phrase could include two or more components of type A (e.g., A1 and A2). In some embodiments, the composition described by this phrase could include two or more components of type B (e.g., B1 and 82). In some embodiments, the composition described by this phrase could include two or more components of type C (e.g., C1 and C2). In some embodiments, the composition described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the composition described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the composition described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating,” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid, Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, hamster, ferret, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

As used herein, the term “bioorthogonal” or “bioorthogonal functional group” refer to a functional group or chemical reaction that can occur inside a living cell, tissue, or organism without interfering with native biological or biochemical processes. A bioorthogonal functional group or reaction is not toxic to cells.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. Ph in Formula I refers to a phenyl group.

The prefix Cₙ-Cₘ preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from 0, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ² where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and —Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless specifically defined, compounds provided herein can also include all isotopes of atoms occurring in the intermediates or final compounds, Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

### Pharmaceutical Compositions

Described herein are pharmaceutical compositions including a compound having Formula I:

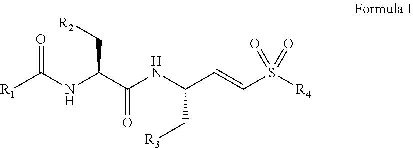

or a pharmaceutically acceptable salt or derivatives thereof,
   wherein
R₁-R₄ are independently a substituted or unsubstituted alkyl, alkenyl, alkynyl, aryl, heteroaryl, or heteroalkyl; and one or more pharmaceutically acceptable carriers. In some embodiments, at least one of R₁, R₂, R₃, and/or R₄ comprise a click motif.

In some embodiments, the compound is present in an effective amount to inhibit a non-viral cysteine protease. In some embodiments, R; is a heteroaryl such as N-methyl-piperazinyl or N-propynyl-piperazinyl. In some embodiments, R₂-R₄ are independently substituted or unsubstituted phenyl, substituted or unsubstituted benzyl, 4-pyridine, or 3-amino-propyl. In some embodiments, R₂-R₄ are substituted or unsubstituted phenyl. In some embodiments, R₂ and R₄ are phenyl. In some embodiments, R₃ is benzyl.

In some embodiments, pharmaceutical compositions including a compound having Formula Ia:

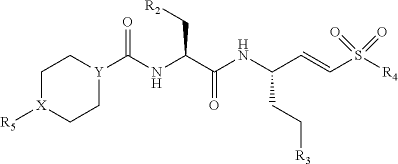

or pharmaceutically acceptable salts thereof, wherein X and Y are independently N, O, or S; R₂-R₅ are independently substituted or unsubstituted alkyl, alkenyl, alkynyl, aryl, heteroaryl, or heteroalkyl; and one or more pharmaceutically acceptable carriers. In some embodiments, at least one of R₁, R₂, R₃, and/or R₅ comprise a click motif.

In some embodiments, the compound is present in an effective amount to inhibit a non-viral cysteine protease. In some embodiments, R₂-R₄ are independently phenyl, substituted phenyl, benzyl, substituted benzyl, 4-pyridine, or 3-amino-propyl. For example, R₂-R₄ are substituted or unsubstituted phenyl. In some embodiments, R₅ is an alkyl such as a methyl, ethyl, propyl, butyl, pentyl, or hexyl. For example, R₅ is a methyl. In some embodiments, R₅ is an alkynyl such as an ethynyl, propynyl, butynyl, pentynyl, or hexynyl. For example, R₅ is a propynyl. In some embodiments, X and Y are N.

In some embodiments, R₅ is -(L¹-CM¹), wherein L¹ is absent, or represents a linking group; and CM¹ represents a first click motif.

In some embodiments, L¹ is absent. In other embodiments, L¹ is present. In some embodiments, L¹ represents a cleavable linker (e.g., a hydrolysable linker, an enzymatically cleavable linker, a photocleavable linker, or a click cleavable linker).

In some embodiments, the compound of Formula I is:

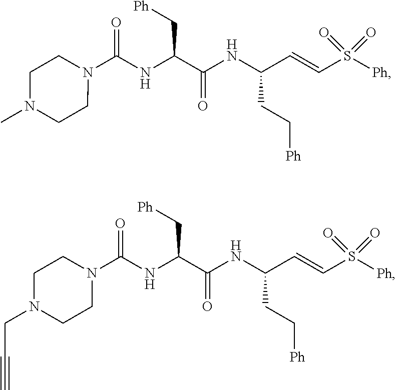 [SMILES: CN1CCN(C(=O)N[C@@H](Cc2ccccc2)C(=O)N[C@H](/C=C/S(=O)(=O)c2ccccc2)CCc2ccccc2)CC1 ; C#CCN1CCN(C(=O)N[C@@H](Cc2ccccc2)C(=O)N[C@H](/C=C/S(=O)(=O)c2ccccc2)CCc2ccccc2)CC1]

or pharmaceutically acceptable salts thereof.

In some embodiments, the non-viral cysteine protease is a mammalian cysteine protease such as cathepsin L. In some embodiments, the non-viral cysteine protease is selected from cruzain, cruzipain, cathepsin L, cathepsin B, cathepsin K, cathepsin S, cathepsin V, or other cysteine proteases. In some embodiments, the compound is a cathepsin L inhibitor.

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

### Method of Identification

Disclosed is a method of identifying a protein target of a compound having Formula I, the method comprising: labeling a protein target with a click target; contacting the labeled protein target with a click complement; and detecting the labeled protein. Also disclosed is a method of identifying a protein target of a compound having Formula I in mammalian cells or tissues infected or not-infected with coronaviruses, the method comprising: labeling a protein target in mammalian cells or tissues infected or not-infected with coronaviruses with a click target; contacting the labeled protein target with a click complement; and detecting the labeled protein. In some embodiments, the protein target is cruzain, cruzipain, cathepsin B, cathepsin L, cathepsin S, or other cysteine proteases.

In some embodiments, the Click Target includes a first click motif. In some embodiments, the Click Target can include a protein target binding moiety conjugated to a first click motif that can participate in a click reaction. In some embodiments, the protein target binding moiety can comprise a functional group capable of chemically reacting with a functional group in the protein target to form a covalent bond.

In some embodiments, the Click Target can be defined by

### Formula I:

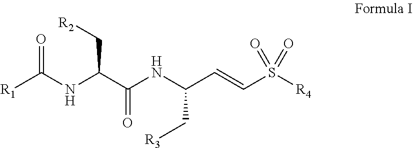 [0104] or a pharmaceutically acceptable salt or derivatives thereof,
wherein
R₁-R₄ are independently a substituted or unsubstituted alkyl, alkenyl, alkynyl, aryl, heteroaryl, or heteroalkyl; wherein at least one of R₁, R₂, R₃, and/or R₄ comprise a click motif. In some embodiments, R₁ is a heteroaryl such as N-methyl-piperazinyl or N-propynyl-piperazinyl. In some embodiments, R₂-R₄ are independently substituted or unsubstituted phenyl, substituted or unsubstituted benzyl, 4-pyridine, or 3-amino-propyl. In some embodiments, R₂-R₄are substituted or unsubstituted phenyl. In some embodiments, R₂ and R₄ are phenyl. In some embodiments, R₃ is benzyl. In some embodiments, R₁ comprises a first click motif.

In some embodiments, the Click Target can be defined by Formula II

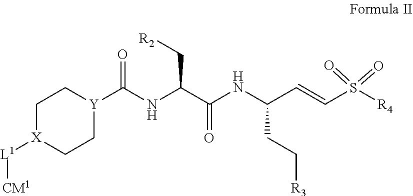

wherein
L¹ is absent, or represents a linking group; and CM¹ represents a first click motif;
X and Y are independently N, O, or S; R₂-R₄ are independently a substituted or unsubstituted alkyl, alkenyl, alkynyl, aryl, heteroaryl, or heteroalkyl. In some embodiments, X and Y are N. In some embodiments, R₂-R₄ are independently substituted or unsubstituted phenyl, substituted or unsubstituted benzyl, 4-pyridine, or 3-amino-propyl. In some embodiments, R₂-R₄ are substituted or unsubstituted phenyl. In some embodiments, R₂ and R₄ are phenyl. In some embodiments, R₃ is benzyl.

In some embodiments, the click target can be a compound of Formula IIa:

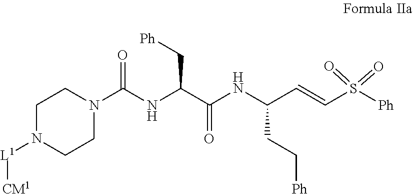

wherein
L¹ is absent, or represents a linking group; and CM¹ represents a first click motif.

In some embodiments, L¹ is absent. In other embodiments, L¹ is present. In some embodiments, L¹ represents a cleavable linker (e.g., a hydrolysable linker, an enzymatically cleavable linker, a photocleavable linker, or a click cleavable linker).

In some embodiments, L1 is alkyl, and CM1 is an alkynyl. In some embodiments, L1 is absent, and CM1 is an alkynyl. In some embodiments, CM1 is a propynyl.

In some embodiments, the Click Complement includes a second click motif bound to detectable moiety. In some embodiments, the Click Complement can be defined by Formula III:

A-L²-CM² Formula III

wherein A represents a detection moiety; L² is absent, or represents a linking group; and CM² represents a second click motif complementary to the first click motif.

In some embodiments, L² is absent. In other embodiments, L² is present. L² represents a cleavable linker (e.g., a hydrolysable linker, an enzymatically cleavable linker, a photocleavable linker, or a click cleavable linker).

The identity of the first click motif and the second click motif are selected, as discussed below, such that the first click motif is capable of chemically reacting with the second click motif to form a covalent bond.

In some examples, the first click motif can comprise a tetrazine (Tz) and the second click motif can comprise an alkene (e.g., a cyclooctene, such as trans-cyclooctene (TCO)). In some examples, the first click motif can comprise an alkene (e.g., a cyclooctene, such as trans-cyclooctene (TCO)) and the second click motif can comprise a tetrazine (Tz).

In other examples, the first click motif can comprise an azide and the second click motif comprises an alkyne (e.g., a cyclooctyne, such as dibenzocyclooctyne (DBCO)).

In other examples, the first click motif can comprise an alkyne (e.g., a cyclooctyne, such as dibenzocyclooctyne (DBCO)) and the second click motif comprises an azide.

In some embodiments, the detection moiety can include a fluorescent probe, chemiluminescent probe, or a phosphorescent probe. In some embodiments, the detection probe is attached to an azide. In some embodiments, the detection moiety can be a fluorescent probe. In some embodiments, the fluorescent probe is attached to an azide. In some embodiments, the fluorescent probe can include, but are not limited to, carboxyrhodamine 110-PEG3-azide (Broadpharm), or cyanine-7-azide (Lumiprobe).

In some embodiments, the first click motif is an alkyne. In some embodiments, the second click motif is an azide.

In some embodiments, the click target is:

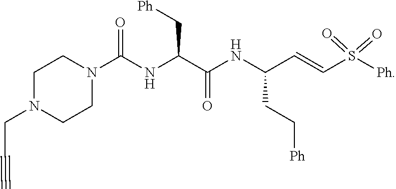 [SMILES: C#CCN1CCN(C(=O)N[C@@H](Cc2ccccc2)C(=O)N[C@H](/C=C/S(=O)(=O)c2ccccc2)CCc2ccccc2)CC1]

### Click Motifs

Example click motif pairs used as the first click motif and the second click motif include, but not limited to, azide with phosphine; azide with cyclooctyne; nitrone with cyclooctyne; nitrile oxide with norbornene; oxanorbornadiene with azide; trans-cyclooctene with s-tetrazine; quadricyclane with bis(dithiobenzil)nickel(II).

In some embodiments, the second click motif comprises an alkene, e.g., a cyclooctene, e.g., a transcyclooctene (TCO) or norbornene (NOR), and the first click motif comprises a tetrazine (Tz). In other embodiments, the second click motif comprises an alkyne, e.g., a cyclooctyne such as dibenzocyclooctyne (DBCO), and the first click motif comprises an azide (Az). In some embodiments, the second click motif comprises a Tz, and the first click motif comprises an alkene such as transcyclooctene (TCO) or norbornene (NOR). Alternatively or in addition, the first click motif comprises an Az, and the second click motif comprises a cyclooctyne such as dibenzocyclooctyne (DBCO). TCO reacts specifically in a click chemistry reaction with a tetrazine (Tz) moiety. DBCO reacts specifically in a click chemistry reaction with an azide (Az) moiety. Norbornene reacts specifically in a click chemistry reaction with a tetrazine (Tz) moiety.

Exemplary click chemistry reactions (and by extension click motifs) are shown below. For example, copper(l)-catalyzed Azide-Alkyne Cycloaddition (CuAAC) comprises using a Copper (Cu) catalyst at room temperature. The Azide-Alkyne Cycloaddition is a 1,3-dipolar cycloaddition between an azide and a terminal or internal alkyne to give a 1,2,3-triazole.

Another example of click chemistry includes Staudinger ligation, which is a reaction that is based on the classic Staudinger reaction of azides with triarylphosphines. It launched the field of bioorthogonal chemistry as the first reaction with completely abiotic functional. The azide acts as a soft electrophile that prefers soft nucleophiles such as phosphines. This is in contrast to most biological nucleophiles which are typically hard nucleophiles. The reaction proceeds selectively under water-tolerant conditions to produce a stable product. Phosphines are completely absent from living systems and do not reduce disulfide bonds despite mild reduction potential. Azides had been shown to be biocompatible in FDA-approved drugs such as azidothymidine and through other uses as cross linkers. Additionally, their small size allows them to be easily incorporated into biomolecules through cellular metabolic pathways.

Copper-free click chemistry is a bioorthogonal reaction first developed by Carolyn Bertozzi as an activated variant of an azide alkyne cycloaddition. Unlike CuAAC, Cu-free click chemistry has been modified to be bioorthogonal by eliminating a cytotoxic copper catalyst, allowing reaction to proceed quickly and without live cell toxicity. Instead of copper, the reaction is a strain-promoted alkyne-azide cycloaddition (SPAAC). It was developed as a faster alternative to the Staudinger ligation, with the first generations reacting over sixty times faster. The incredible bioorthogonality of the reaction has allowed the Cu-free click reaction to be applied within cultured cells, live zebrafish, and mice. Cyclooctynes were selected as the smallest stable alkyne ring which increases reactivity through ring strain which has calculated to be 19.9 kcal/mol.

Copper-free click chemistry also includes nitrone dipole cycloaddition. Copper-free click chemistry has been adapted to use nitrones as the 1,3-dipole rather than azides and has been used in the modification of peptides.

This cycloaddition between a nitrone and a cyclooctyne forms N-alkylated isoxazolines. The reaction rate is enhanced by water and is extremely fast with second order rate constants ranging from 12 to 32 M⁻¹·s⁻¹, depending on the substitution of the nitrone. Although the reaction is extremely fast, incorporating the nitrone into biomolecules through metabolic labeling has only been achieved through post-translational peptide modification.

Another example of click chemistry includes norbornene cycloaddition. 1,3 dipolar cycloadditions have been developed as a bioorthogonal reaction using a nitrile oxide as a 1,3-dipole and a norbornene as a dipolarophile. Its primary use has been in labeling DNA and RNA in automated oligonucleotide synthesizers.

Norbornenes were selected as dipolarophiles due to their balance between strain-promoted reactivity and stability. The drawbacks of this reaction include the cross-reactivity of the nitrile oxide due to strong electrophilicity and slow reaction kinetics.

Another example of click chemistry includes oxanorbornadiene cycloaddition. The oxanorbornadiene cycloaddition is a 1,3-dipolar cycloaddition followed by a retro-Diels Alder reaction to generate a triazole-linked conjugate with the elimination of a furan molecule. This reaction is useful in peptide labeling experiments, and it has also been used in the generation of SPECT imaging compounds.

Ring strain and electron deficiency in the oxanorbornadiene increase reactivity towards the cycloaddition rate-limiting step. The retro-Diels Alder reaction occurs quickly afterwards to form the stable 1,2,3 triazole. Limitations of this reaction include poor tolerance for substituents which may change electronics of the oxanorbornadiene and low rates (second order rate constants on the order of 10⁻⁴).

Another example of click chemistry includes tetrazine ligation. The tetrazine ligation is the reaction of a trans-cyclooctene and an s-tetrazine in an inverse-demand Diels Alder reaction followed by a retro-Diels Alder reaction to eliminate nitrogen gas. The reaction is extremely rapid with a second order rate constant of 2000 M⁻¹·s⁻¹ (in 9:1 methanol/water) allowing modifications of biomolecules at extremely low concentrations.

The highly strained trans-cyclooctene is used as a reactive dienophile. The diene is a 3,6-diaryl-s-tetrazine which has been substituted in order to resist immediate reaction with water. The reaction proceeds through an initial cycloaddition followed by a reverse Diels Alder to eliminate N₂ and prevent reversibility of the reaction.

Not only is the reaction tolerant of water, but it has been found that the rate increases in aqueous media. Reactions have also been performed using norbornenes as dienophiles at second order rates on the order of 1 M⁻¹·s⁻¹ in aqueous media. The reaction has been applied in labeling live cells and polymer coupling.

Another example of click chemistry includes is [4+1] cycloaddition. This isocyanide click reaction is a [4+1] cycloaddition followed by a retro-Diels Alder elimination of N₂.

The reaction proceeds with an initial [4+1] cycloaddition followed by a reversion to eliminate a thermodynamic sink and prevent reversibility. This product is stable if a tertiary amine or isocyanopropanoate is used. If a secondary or primary isocyanide is used, the produce will form an imine which is quickly hydrolyzed.

Isocyanide is a favored chemical reporter due to its small size, stability, non-toxicity, and absence in mammalian systems. However, the reaction is slow, with second order rate constants on the order of 10⁻² M⁻¹·s⁻¹.

Another example of click chemistry includes quadricyclane ligation. The quadricyclane ligation utilizes a highly strained quadricyclane to undergo [2+2+2]cycloaddition with TT systems.

Quadricyclane is abiotic, unreactive with biomolecules (due to complete saturation), relatively small, and highly strained (^{˜}80 kcal/mol). However, it is highly stable at room temperature and in aqueous conditions at physiological pH. It is selectively able to react with electron-poor π systems but not simple alkenes, alkynes, or cyclooctynes.

Bis(dithiobenzil)nickel(II) was chosen as a reaction partner out of a candidate screen based on reactivity. To prevent light-induced reversion to norbornadiene, diethyldithiocarbamate is added to chelate the nickel in the product.

These reactions are enhanced by aqueous conditions with a second order rate constant of 0.25 M⁻¹·s⁻¹. Of particular interest is that it has been proven to be bioorthogonal to both oxime formation and copper-free click chemistry. The exemplary click chemistry reactions have high specificity, efficient kinetics, and occur in vivo under physiological conditions(see, e.g., Baskin et al. Proc. Natl. Acad. Sci. USA 104(2007):16793; Oneto et al. Acta biomaterilia (2014); Neves et al. Bioconjugate chemistry 24(2013):934; Koo et al. Angewandte Chemie 51(2012):11836; and Rossin et al. Angewandte Chemie 49(2010):3375. For a review of a wide variety of click chemistry reactions and their methodologies, see e.g., Nwe K and Brechbiel M W, 2009 Cancer Biotherapy and Radiopharmaceuticals, 24(3): 289-302; Kolb H C et al., 2001 Angew. Chem, Int. Ed. 40: 2004-2021. The entire contents of each of the foregoing references are incorporated herein by reference.

Exemplary click motif pairs are shown in the table below.

| Functional group/ | Paired | | Reaction type |
|---|---|---|---|
| Click Motif | with | Functional group/Click Motif | (Reference) |
| Azide | | Phosphine | Staudinger |
| | | | ligation (Saxon |
| | | | et al. Science |
| | | | 287(2000): |
| | | | 2007-10) |
| azide | | Cyclooctyne, e.g., dibenzocyclooctyne, one | Copper-free |
| | | of the cyclooctynes shown below, or other | click chemistry |
| | | similar cyclooctynes: | (Jewett et al. |
| | | 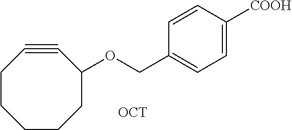 | J. Am. Chem. Soc.132.11 (2010):3688- 90; Sletten et al. Organic Letters 10.14 (2008):3097-9; Lutz. Angew. |
| | | | Chem., Int. Ed |
| | | | 47.12(2008): |
| | | | 2182) |
| | | 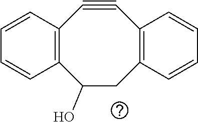 [SMILES: OC1Cc2ccccc2C#Cc2ccccc21] | |
| | | 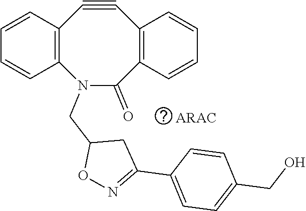 [SMILES: O=C1c2ccccc2C#Cc2ccccc2N1CC1CC(c2ccc(CO)cc2)=NO1] | |
| | | 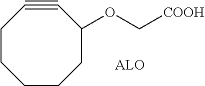 [SMILES: O=C(O)COC1C#CCCCCC1] | |
| | | 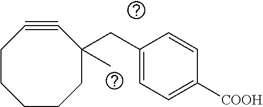 [SMILES: CC1(Cc2ccc(C(=O)O)cc2)C#CCCCCC1] | |
| | | 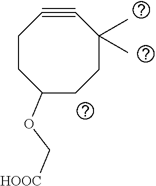 | |
| | | 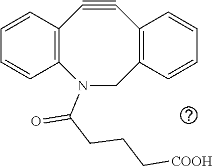 [SMILES: O=C(O)CCCC(=O)N1Cc2ccccc2C#Cc2ccccc21] | |
| | | 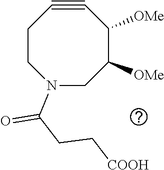 [SMILES: CO[C@H]1C#CCCN(C(=O)CCC(=O)O)C[C@@H]1OC] | |
| Nitrone | | Cyclooctyne | Nitrone Dipole |
| | | | Cycloaddition |
| | | | (Ning et al. |
| | | | Angew. Chem., |
| | | | Int. Ed |
| | | | 49.17(2010): |
| | | | 3065) |
| Nitrile oxide | | Norbornene | Norbornene |
| | | | Cycloaddition |
| | | | (Gutsmiedl et al. |
| | | | Organic Letters |
| | | | 11.11(2009): |
| | | | 2405-8) |
| Oxanorbornadiene | | Azide | Oxanorbornadiene |
| | | | Cycloaddition |
| | | | (Van Berkel et al. |
| | | | ChemBioChem |
| | | | 8.13(2007):1504-8) |
| Trans-cyclooctene, | | s-tetrazine | Tetrazine ligation |
| norbornene, or | | | (Hansell et al. |
| other alkene | | | J. Am. Chem. Soc. |
| | | | 133.35(2011): |
| | | | 13828-31) |
| Nitrile | | 1,2,4,5-tetrazine | [4 + 1] cyclo- |
| | | | addition (Stackman |
| | | | et al. Organic and |
| | | | Biomol. Chem. |
| | | | 9.21(2011):7303) |
| Quadricyclane | | Bis(dithiobenzil)nickel(II) | Quadricyclane |
| | | | Ligation (Sletten |
| | | | et al. J. Am. |
| | | | Chem. Soc. |
| | | | 133.44(2011): |
| | | | 17570-3 |
| Ketone or | | Hydrazines, hydrazones, oximes, amines, ureas, | Non-aldol carbonyl |
| aldehyde | | thioureas, etc. | chemistry |
| | | | (Khomyakova |
| | | | EA, et al. |
| | | | Nucleosides |
| | | | Nucleotides |
| | | | Nucleic Acids. |
| | | | 30(7-8) |
| | | | (2011)577-84 |
| Thiol | | Maleimide | Michael addition |
| | | | (Zhou et al. |
| | | | Bioconjug Chem |
| | | | 2007 18(2):323-32.) |
| Dienes | | Dienophiles | Diels Alder |
| | | | (Rossin et al. |
| | | | Nucl Med. (2013) |
| | | | 54(11):1989-95) |
| Tetrazine | | norbornene, propene, trans-cyclooctene, other | |
| | | strained alkenes. | |

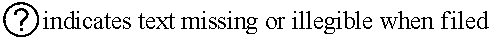

Other suitable include the motifs can be found, for example, in Patterson, D. M., et al. “Finding the Right (Bioorthogonal) Chemistry,” ACS Chem. Biol. 2014, 9(3): 592-605; Akgun, B., et al. “Synergic “Click” Boronate/Thiosemicarbazone System for Fast and Irreversible Bioorthogonal Conjugation in Live Cells,” J. Am Chem. Soc., 2017, 139(40):14285-14291; and Akgun, B. and Hall, D. G. “Fast and Tight Boronate Formation for Click Bioorthogonal Conjugation,” Angew. Chem., Int. Ed. 2016, 55(12): 3909-3913, each of which is hereby incorporated by reference in its entirety.

### Linking Groups

When present, the linking group can be any suitable group or moiety which is at minimum bivalent, and connects the two radical moieties to which the linking group is attached in the compounds described herein. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprises one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

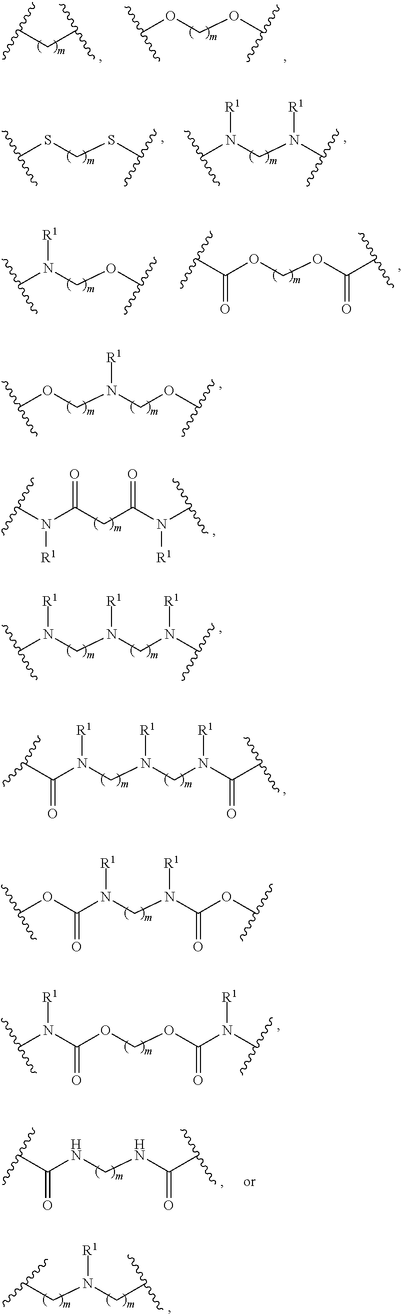

where m is an integer from 1 to 12 and R¹ is, independently for each occurrence, hydrogen, an alkyl group, an aryl group, or a heterocyclic group.

If desired, the linker can serve to modify the solubility of the compounds described herein. In some embodiments, the linker is hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photo-cleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel). In other embodiments, the linker can be “click cleavable” (i.e., a click-to-release linker). Such linkers are cleaved when a click motif to which the linker is bound participates in a click reaction. Examples of click cleavable linkers (and associated click motifs) are known in the art. See, for example, Versteegen et al. Angew. Chem. Int. Ed., 2018, 57(33): 10494-10499; Versteegen et al. Angew. Cher Int. Ed., 2013, 52(52): 14112-14116; U.S. Patent Application Publication No. 2019/0247513; and U.S. Pat. No. 10,004,810; each of which is hereby incorporated by reference in its entirety. In embodiments where an external stimulus (e.g., irradiation by light or application of a magnetic field) induces cleavage, the methods described herein can further comprise the step of applying the external stimulus to induce cleavage. In other embodiments, the linker can be non-cleavable.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of an infection with a Mycobacterium.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. WV. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid, Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, I,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the compound as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, doluitegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type 1, interferon type 11, interferon type Ill, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of additional active agents include chloroquine, hydrochloroquine, Vitamin D, and Vitamin C

In some embodiments, the compound as used in the methods described herein may be administered in combination or alternation with one or more anticytokine or immunomodulatory agents, representative examples of which include, but are not limited to, tocilizumab, sarilumab, bevacizumab, fingolimod, imiquimod, and eculizumab.

In some embodiments, the compound as used in the methods described herein may be administered in combination or alternation with an immunoglobulin therapy.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The present disclosure can be better understood by reference to the following examples which are offered by way of illustration. The disclosure is not limited to the examples given herein.

### Abstract

K11777, a di-peptide analog that contains an electrophilic vinyl-sulfone group, acts as an exceptionally potent, covalent inactivator of cruzain and cathepsins B, L, and S, but does not inhibit or inactivate either the Main protease (3CL protease) or papain-like protease of SARS CoV-2. Upon treatment with 0.08-0.60 mM K777, potent inhibition of the coronavirus-induced cytopathic effect caused by SARS-CoV-2 infection of Vero E6 and A549/ACE2 cells. No toxicity was observed in host cells at concentrations of 100 TIM. The protein targets of K1777 in virally-infected and non-infected cell lines may be visualized and identified using 4N-propargyl-K11777, as a covalent, bio-orthogonal protein-labeling agent. Use of 4N-propargyl-K11777 has identified a host-cell protein of the same molecular weights as mammalian cathepsin L and cathepsin B.

### INTRODUCTION

Data demonstrated that the clinical-stage compound K11777, an irreversible covalent inactivator of cathepsin L and cruzain, also potently blocks SARS-CoV-2 infection of Vero E6 (monkey kidney epithelial cells) and A549/ACE2 (human lung epithelial cells expressing the ACE2 receptor). K11777 has recently been progressed as a pre-investigational new drug, sponsored by Selva Therapeutics, for the treatment of Chagas disease. Consequently, detailed data of its pharmacology, pharmacokinetics, tissue distribution, safety and toxicology are known. K11777 could therefore be rapidly progressed to human clinical trials for the treatment of COVID-19.

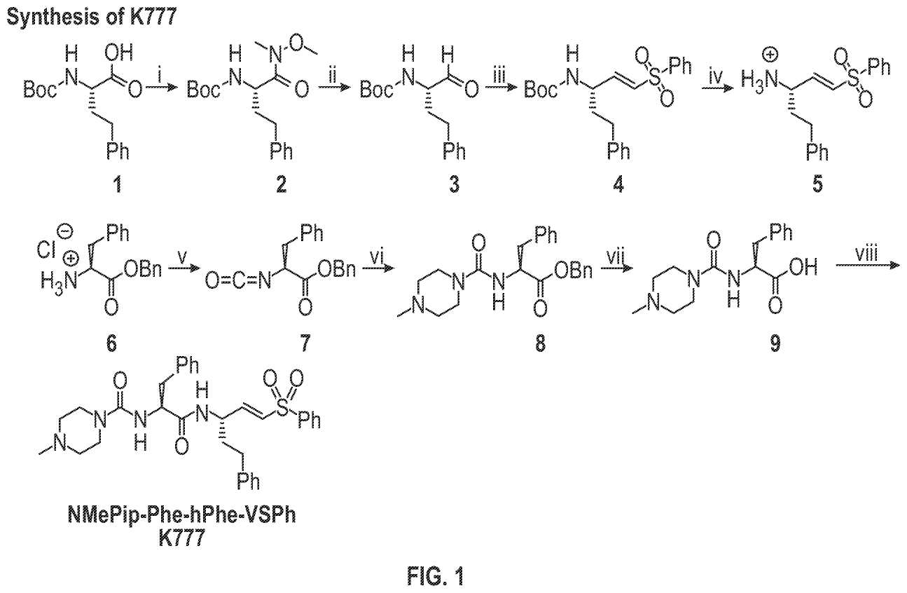

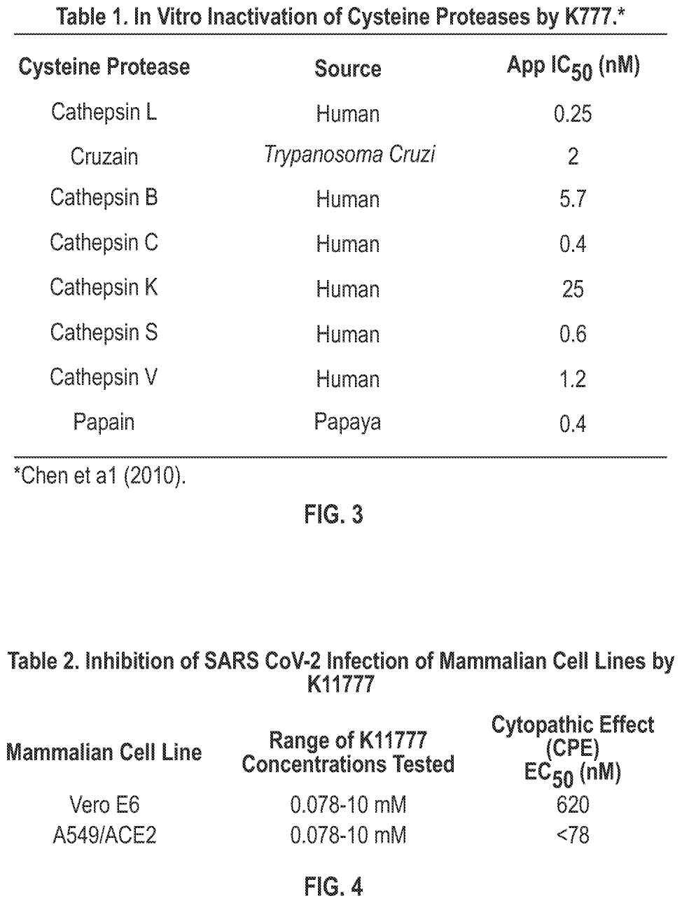

K11777 (also called K777, S-001, or SLV-213) with the chemical name of 4-methyl-N-((S)-1-oxo-3-phenyl-1-(((S)-1-phenyl-5-(phenylsulfonyl)pentan-3-yl)amino)propan-2-yl)piperazine-1-carboxamide; (see FIG. 1) is a highly potent, irreversible, covalent inactivator of mammalian cathepsin L and other cysteine proteases of clan CA. K11777 inactivates several mammalian, including human, cysteine proteases (FIG. 3), and is particularly effective vs. cruzain and human cathepsins L, and S. Due to its excellent activity vs. cruzain, an essential cysteine protease of Trypanosoma cruzi, the causative agent of Chagas disease, the laboratories of Professor James H. McKerrow in collaboration with Selva Therapeutics have progressed K11777 to the initiation of an IND for the treatment of Chagas disease, and as a result, there is an extensive amount of pre-clinical data amassed for this compound.

In 2005, Zhou and colleagues showed that K11777 was an exceptionally potent inhibitor of the infection of Vero, A549, and other mammalian cells which are susceptible to infection by a pseudovirus SARS CoV-1. Cathepsin L was implicated as a target of K11777 in host cells, and its inactivation by K11777 contributed to the anti-viral effect. This results are similar to those of other studies in which other, more generic inhibitors of cysteine proteases also implicated cathepsin L as a host-cell enabling factor in COV-1 infectivity. We have recently shown that the same is true for SARS CoV-2 in Vero E6 and A549 cells bearing the ACE2 receptor (A549/ACE2). These preliminary results suggest that K11777 may be a highly effective therapy for the treatment of COVID-19.

Yang et al have shown that the N4-propargyl analogue of K11777 (R=alkynyl) is a bio-orthogonal label for the visualization, extraction and sequencing of cellular protein targets of K11777.

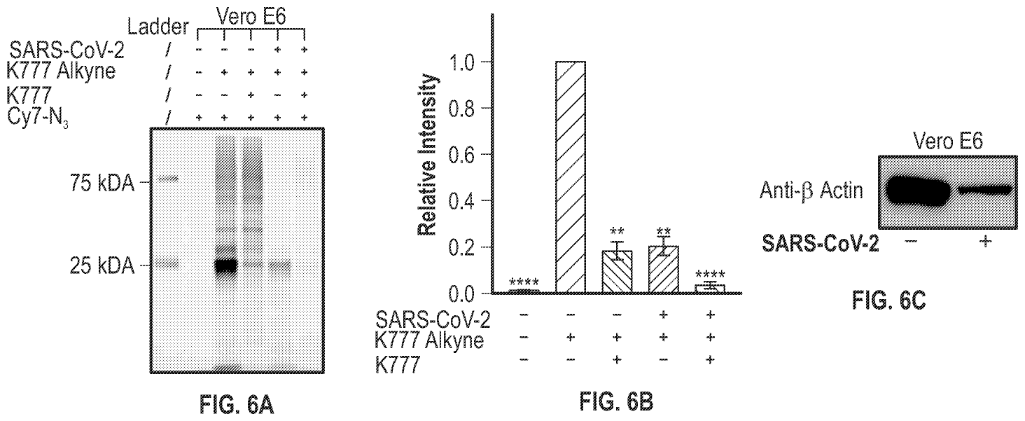

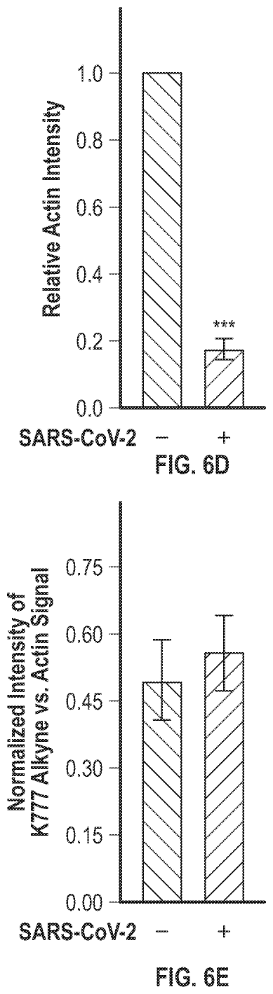

We have used the N4-propargyl analogue of K1177 (K11777-alkyne) to label proteins in both CoV-2-infected and un-infected Vero E6 cells. The alkynyl group was reacted with cyanine-7-azide in order to render covalently-labeled proteins in the cells fluorescence. K11777 was added for some samples as a pre-treatment to label protein targets prior to reaction with K11777-alkyne in order to determine those cellular or viral proteins that are specifically labeled by K11777 (FIG. 6).

The alkyne substituent of the N4-propargyl analogue of K11777 was also reacted with an insoluble bead bearing an azide substituent in order to enrich labeled proteins. Captured proteins were cleaved off the bead using trypsin, and the resulting peptides were sequenced by mass spectrometry.

N4-propargyl analogue of K11777 labels only cellular proteins of an apparent molecular weight of 24-25 kDa in Vero E6 cells (FIG. 6), and no additional proteins were detected in Vero E6 cells that had been infected with CoV-2.

Quantification of the mammalian protein actin in the infected and un-infected Vero E6 cell lysates indicated greatly reduced levels of mammalian proteins in the virally-infected samples. Normalization of the protein levels between the CoV-2-infected and un-infected cell samples revealed that the extent of the labeling of the 24/25 kDa protein by the N4-propargyl analogue of K11777 was similar.

Pre-treatment of cells with K11777 prevented labelling of the 24-25-kDa protein.

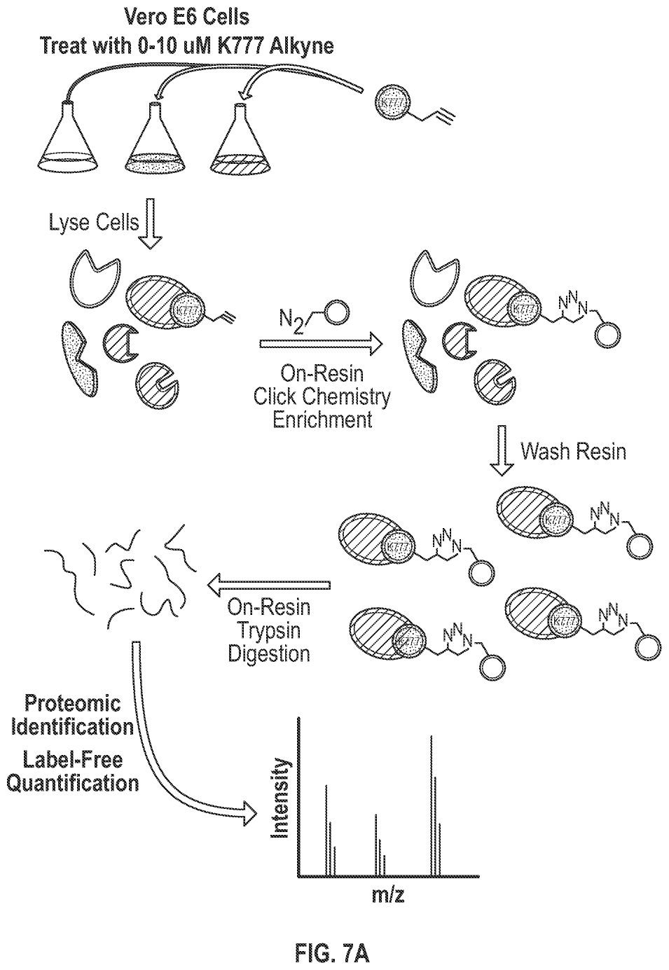

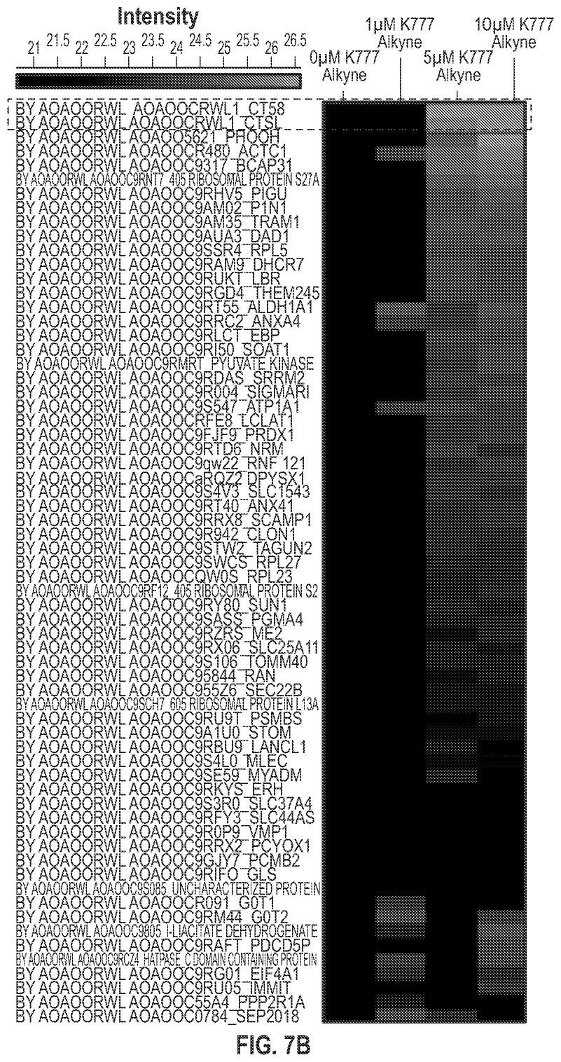

Sequencing of the bead-captured proteins demonstrated that the most enriched proteins labeled with the N4-propargyl analogue of K11777 in Vero E6 samples were cathepsins L and B (FIG. 7).

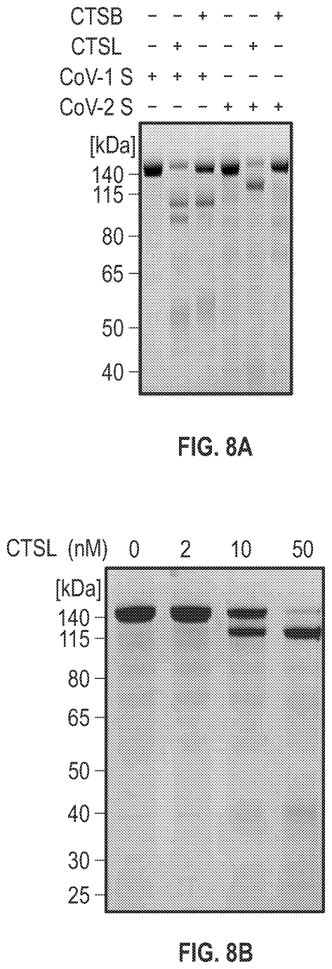

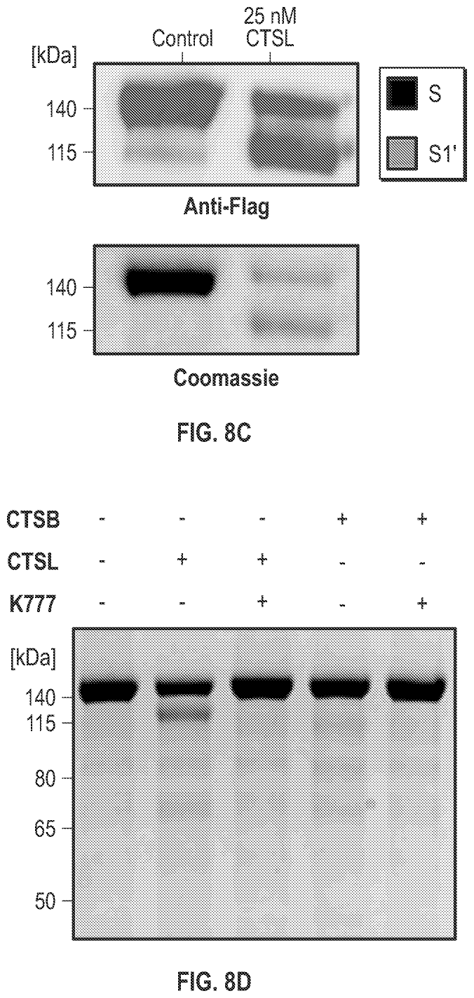

Treatment of purified SARS CoV-2 Spike protein demonstrated proteolysis of this protein into two protein bands by 250 nM of cathepsin L, which was blocked by added K11777. The protein fragments were different in size than those obtained by treatment of the SARS CoV-1 Spike protein by added cathepsin S (FIG. 8). Cathepsin B did not proteolyze purified SARS CoV-2 Spike protein under identical conditions.

Cathepsin B can catalyze cleavage of SARS CoV-1 Spike protein.

### Abbreviations Used

DIPEA, N,N-Diisopropylethylamine; hPhe, homophenylalanine; LAH, lithium aluminum hydride; LHMDS, lithium bis(trimethylsilyl)amide; NMePip, N-methylpiperazinyl; T3P, propylphosphonic anhydride; TFA, trifluoroacetic acid; VSPh, vinyl sulfone phenyl, AMC, 7-amino-4-methylcoumarin; CHAPS, 3-[3-(cholamidopropyl)dimethylammonio]-1-propanesulfonate; and MES, 2-(N-morpholino)ethanesulfonic acid.

### Example 1

### Preparation of K11777, N4-Propargyl-K11777 and Abz-Ser-Ala-Val-Leu-Gln*Ser-Gly-Phe-Arg-Lys-NH₂

The reagents and starting materials used were obtained from commercial vendors and used as received without any purification. Reactions were carried in an inert atmosphere of nitrogen unless otherwise specified. Progress of the reactions were monitored using Thin Layer Chromatography (TLC) and LC-MS analysis, by employing an HPLC-MS (UltiMate 3000 equipped with a diode array coupled to a MSQ Plus Single Quadrupole Mass Spectrometer, ThermoFisher Scientific) using electrospray positive and negative ionization detectors. HPLC conditions used: column: Phenomenex Luna 5 mm C18(2) 100 Å, 4.6 mm, 50 mm, Mobile phase A: water with 0.1% formic acid (v/v). Mobile phase B: MeCN with 0.1% formic acid (v/v). Temperature: 25° C. Gradient: 0-100% B over 6 min, then a 2 min hold at 100% B. Flow: 1 mL/min. Detection: MS and UV at 254, 280, 214, and 350 nm. ¹H/¹³C NMR spectra were obtained in CDCl₃, CD₃OD, or DMSO-d₆ at 400 MHz/100 MHz at 298 K on a Bruker Avance III NanoBay console with an Ascend magnet. The following abbreviations were utilized to describe peak patterns when appropriate: br=broad, s=singlet, d=doublet, q=quartet, t=triplet, and m=multiplet. The final compounds used for testing in assays and biological studies had purities that were determined to be >95% as evaluated by their proton NMR spectra and/or their HPLC/MS traces based on ultraviolet detection at 254 nm (K777 and K777 alkyne) or 350 nm (Abz FRET peptide).

All reagents and starting materials were obtained from commercial suppliers and used without further purification unless otherwise stated. Solution phase reactions were conducted under an atmosphere of nitrogen at ambient temperature unless otherwise noted. Reaction progress was monitored using thin-layer chromatography and by HPLC-MS (UltiMate 3000 equipped with a diode array coupled to an ISQ EM single quadrupole mass spectrometer, Thermo Fisher Scientific) using electrospray positive and negative ionization detectors. Reported liquid chromatography retention times (t_{R}) were established using the following conditions: column: Phenomenex Luna 5 μm C18(2) 100 Å, 4.6 mm, 50 mm; mobile phase A: water with 0.1% formic acid (v/v): mobile phase B: MeCN with 0.1% formic acid (v/v); temperature: 25° C.; gradient: 0-100% B over 6 min, then a 2 min hold at 100% B; flow: 1 mL min⁻¹; and detection: MS and UV at 254, 280, 214, and 350 nm.

Semi-preparative HPLC purification of compounds was performed on a Thermo Fisher Scientific UltiMate 3000 with a single wavelength detector coupled to a fraction collector. Purifications were conducted using the following conditions: column: Phenomenex Luna 5 μm C18(2) 100 Å, 21.2 mm, 250 mm; mobile phase A: water with 0.1% formic acid (v/v); mobile phase B: acetonitrile with 0.1% formic acid (v/v); temperature: room temperature.

### Example 2

### Fmoc-Lys(DNP)-OH

A round bottom flask charged with Fmoc-Lys-OH (2 g, 5.43 mmol) was purged with N₂ gas to yield positive pressure, followed by the addition of 50 mL anhydrous dichloromethane. Subsequently, DIPEA (2.84 mL, 16.3 mmol) was added, followed by the dropwise addition of 1-fluoro-2,4-dinitrobenzene (700 μL, 5.54 mmol). The mixture was allowed to react for 3.5 hours at which time the reaction mixture was washed with 1N HCl (1 time), water (3 times), brine (1 time), dried over Na₂SO₄, and concentrated under reduced pressure. The crude product was subjected to flash purification on silica (2% MeOH:DCM), which following concentration under reduced pressure yielded a bright yellow fluffy powder (1.89 g, 65.2% yield). LC-MS t_{R}: 5.99 min, m/z 535.12 [M+1H], C₂₇H₂₆N₄O₈ Calcd. 535.18 [M+1H].

### Example 3

### Boc-2-Abz-OH

A round bottom flask charged with 2-aminobenzoic acid (2 g, 14.58 mmoles) was dissolved in 20 mL of water and the pH was adjusted to 8 by adding 10 N NaOH dropwise. Subsequently, di-tert-butyl dicarbonate (3.5 g, 16.04 mmol) was dissolved in 20 mL of anhydrous THF was added dropwise to the reaction which was allowed to proceed overnight. The organic was removed under reduced pressure the aqueous layer was acidified with 2N HCL and extracted with ethyl acetate 3 times. The organic layer was washed with water (2 times), and brine (2 times) and dried over dried over anhydrous sodium sulfate and removed under reduced pressure yielding an off-white powder. (2.76 g, 80.7% yield) LC-MS t_{R}: 5.36 min, m/z 236.08 [M−1H], C₁₂H₁₅NO₄ Calcd. 236.10 [M−1H].

### Example 4

### Fmoc-SAVLQSGFRK(DNP)-NH₂

To a syringe fitted with a frit, 200 umol of Rink Amide AM resin (Novabiochem, 0.73 mmol/g) was washed with dichloromethane (DCM) and swelled in N,N-dimethylformamide (DMF). The Fmoc resin was deprotected using 20% piperidine in DMF (v/v) for 15 minutes (3 times), followed by five washes with DMF. Coupling of Fmoc-Lys(DNP)-OH was conducted using 3-told excess of the amino acid versus resin loading (0.6 mmol), (1-Cyano-2-ethoxy-2-oxoethylidenaminooxy)dimethylamino-morpholino-carbenium hexafluorophosphate (COMU) (0.6 mmol), and DIPEA (1.2 mmol) under agitation for 3 hours (2 times). Washing of the resin with DMF, followed by ninhydrin analysis verified the coupling was complete. The resin was then capped by 45 minutes of agitation with 25% acetic anhydride in DMF (v/v) and DIEA (0.3 mmol), followed by DMF washes (5 times). Subsequent synthesis of the peptide occurred in a stepwise fashion. deprotection of the Fmoc protecting group by 20% piperidine in DMF (v/v) for 15 minutes (2 times), DMF wash (5 times), ninhydrin analysis, addition of Fmoc-AA-OH (0.6 mmol), COMU (0.6 mmol), and DIEA (1.2 mmol) (allowed to pre-activate for 2 minutes prior to addition to resin) and couple for 1 hour, wash resin with DMF (5 times), ninhydrin analysis, cap unreacted amine groups with 25% acetic anhydride in DMF (v/v) and DIPEA (0.3 mmol) for 15 minutes, wash resin with DMF (5 times). All steps were carried out under constant agitation. Test cleavage of the peptide was conducted using ˜5-10 mg of resin suspended in hexafluoroisopropanol (HFIP) containing 0.5 N HCl (aq), 5% v/v water, and 2.5% v/v triisopropylsilane for two hours. The solution was then removed under a stream of N₂ gas and the resulting material was dissolved in acetonitrile, filtered, and analyzed by LC-MS, showing that the peptide was the major product (m/z 740.4 [M+2H]; 1479.7 [M+1H] or 740.4 [M+2H] calcd.). The peptide-laden resin was then washed with DCM (3 times), dried under vacuum, and stored in a desiccator for further synthesis.

### Example 5

### Abz-SAVLQSGFRK(DNP)-NH₂

To a syringe fitted with a frit, half of dry resin bound Fmoc-SAVLQSGFRK(DNP)-Rink Amide peptide was suspended in DMF for 20 minutes. Treatment with 20% piperidine in DMF (v/v) for 15 minutes (3 times), followed by DMF washes (5 times) and a ninhydrin test confirmed removal of the Fmoc group. To the free amine. Boc-2-Abz-OH (0.3 mmol). COMU (0.3 mmol), and DIPEA (0.6 mmol) (allowed to pre-activate for 2 minutes) was added and allowed to couple overnight. The resin was washed with DMF (5 times) and the coupling was repeated for another four hours to enhance the yield. The resin was then washed with DMF (5 times), DCM (3 times), and MeOH (3 times). Subsequent cleavage of the peptide with HFIP containing 0.5 N HCl (aq), 5% v/v water, and 2.5% v/v triisopropylsilane for four hours (2 times) was followed by removal of the solution via rotary evaporator and precipitation of the peptide in ice cold diethyl ether. The crude mixture was solubilized in a small volume of DMF and subjected to semi-preparative HPLC (5%-52% B over 20 mins, then 52-100% B to 26 minutes, and 100% B until 29 minutes at 21.2 ml/min). The fractions containing pure peptide (≥95% pure) were then lyophilized to yield a fluffy yellow powder (42.4 mg, 29.2% yield as a formic acid salt) LC-MS t_{R}: 5.36 min, m/z 688.89 [M+2H], 459.79 [M+3H], C₆₁H₈₉N₁₉O₁₈ Calcd 1376.66 [M+1H], 688.83 [M+2H], 459.56 [M+3H].

### Example 7

### Synthesis of K11777

Conditions and reagents: (i) N,O-dimethylhydroxylamine hydrochloride, T3P, DIPEA, DCM, 0° C., 30 min; (ii) LAH, THF, −10° C., 30 min; (iii) Diethyl (phenylsulfonyl)methane phosphonate, LHMDS, THF, −10° C., 20 min, 0° C., 1 h; (iv) TFA, DCM, 0° C., 3 h; (v) a) DCM, Sat. aq. NaHCO₃, b) triphosgene, 0° C., 15 min; (vi) 1-Methylpiperazine, THF, DIPEA, 0° C., 10 min, 25° C., 16 h; (vii) 10% Pd/C, MeOH, 25° C., 20 h; (viii) 5, T3P, DIPEA, DCM, −10° C., 10 min, 0° C., 1 h.

Boc-protected L-homophenylalanine 1 was purchased from a commercial source, converted to Weinreb amide by T3P-catalyzed coupling to N,O-dimethylhydroxylamine hydrochloride to afford 2. Reduction of the Weinreb amide 2 using LAH at −10° C. in anhydrous THF provided the Boc-L-homophenylalanine aldehyde 3. Homer-Wadsworth-Emmons reaction of the aldehyde 3 with the commercially available Diethyl (phenylsulfonyl)methane phosphonate yielded the vinyl phenyl sulfone 4.

L-Phenylalanine benzyl ester hydrochloride 6 was reacted with triphosgene to give Isocyanate 7, which was further reacted with 1-Methylpiperazine to give NMe-Pip-Phe-OBn 8. NMe-Pip-Phe-OBn was subjected to debenzylation using 10% Pd/C with H₂ gas to give NMe-Pip-Phe-OH 9. TFA Deprotection of the Boc group of the vinyl phenyl sulfone 4 gave the TFA salt 5. The T3P-catalyzed coupling of 5 with NMe-Pip-Phe-OH 9 gave the required compound K11777 (see FIG. 1).

### Example 8

### Synthesis of N4Propargyl-K11777

Conditions and reagents: (v) a) DCM, Sat. aq. NaHCO₃, b) triphosgene, 0° C., 15 min; (viii) 5, T3P, DIPEA, DCM, −10° C., 10 min, 0° C., 1 h; (ix) propargyl bromide, DIPEA, CHCl₃, −10° C., 30 min, 25° C., 18 h; (x) TFA, DCM, 0° C., 3 h, 25° C., 1 h; (xi) 12, THF, DIPEA, 0° C., 1 h, 25° C., 16 h; (xii) a) LiOH, THF, H₂O, 0° C., 6 h, b) 4N HCl in dioxane to pH 2.

Commercially available t-butyl Piperazine-1-carboxylate (1-N-Boc-piperazine) 10 was reacted with Propargyl bromide to give 1-N-Boc-Propargyl-Piperazine 11, which was further treated with TFA to give 1-N-Propargyl-Piperazine TFA salt 12.

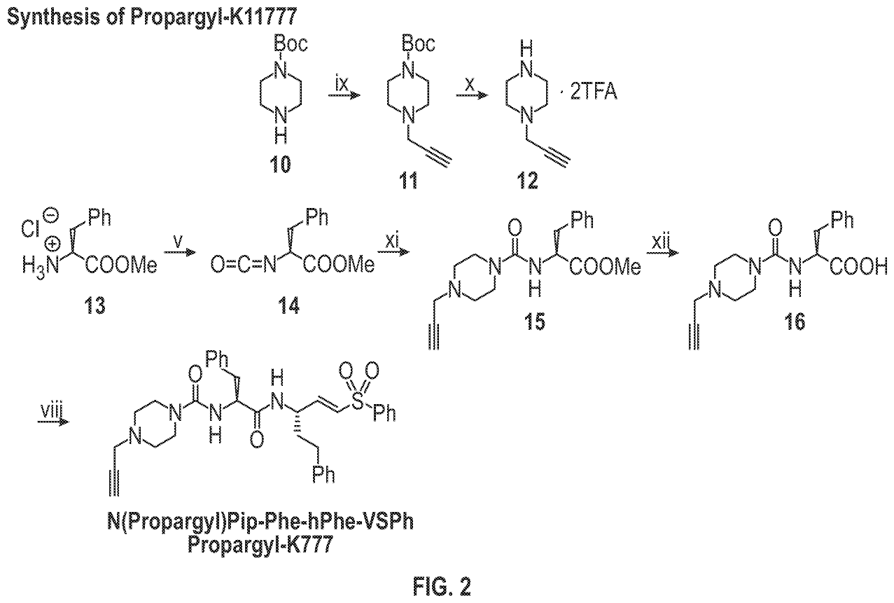

L-phenylalanine methyl ester hydrochloride was reacted with triphosgene to give Isocyanate 14, which was further reacted with 12 to give N-Propargyl-Pip-Phe-OMe 15. The compound 15 was subjected to LiOH hydrolysis to give N-Propargyl-Pip-Phe-OH 16. T3P-catalyzed coupling of 5 with acid 16 gave the required compound Propargyl-K777 (see FIG. 2).

### Example 9

### 4-methyl-N-((S)-1-oxo-3-phenyl-1-(((S,E)-5-phenyl-1-(phenylsulfonyl)pent-1-en-3-yl)amino)propan-2-yl)piperazine-1-carboxamide (K11777, NMePip-Phe-hPhe-VSPh)

To a suspension of TFA salt 5 (0.100 g, 0.240 mmol) in DCM (5 mL) at −10° C., was added dropwise DIPEA (0.48 mL, 2.744 mmol), followed by addition of the acid 9 (0.100 g, 0.343 mmol) and dropwise addition of T3P (0.33 mL, 0.515 mmol). The reaction was continued at the same temperature for 10 min and 0° C. for 1 h. Upon the completion of the reaction as revealed by TLC analysis (MeOH/DCM=1:10, v/v), the reaction mixture was diluted with DCM (50 mL), and then washed with Sat. aq. NaHCO₃ (1×), H₂O (3×) and brine (1×). The organic layer was dried over anh. Na₂SO₄ and filtered. The filtrate was concentrated in vacuo to afford the crude product, which was purified by silica gel column chromatography using a gradient of 1%-10% of MeOH in DCM as eluent to yield the pure product 4-methyl-N-((S)-1-oxo-3-phenyl-1-(((S,E)-5-phenyl-1-(phenylsulfonyl)pent-1-en-3-yl)amino)propan-2-yl)piperazine-1-carboxamide (K11777, NMePip-Phe-hPhe-VSPh, White solid, 0.089 g, 0.155 mmol, 45% yield). ¹H NMR (400 MHz, CDCl₃) δ 1.62-1.87 (m, 2H), 2.22 (s, 3H), 2.23-2.29 (m, 4H), 2.42-2.57 (m, 2H), 2.94-3.05 (m, 2H), 3.22-3.35 (m, 4H), 4.52-4.68 (m, 2H), 5.26 (d, 1H, J=7.8 Hz), 6.12 (dd, 1H, J, =1.5 Hz, J₂=15.1 Hz), 6.78 (dd, 1H, J, =5.0 Hz, J₂=15.1 Hz), 6.98 (d, 2H, J=7.0 Hz), 7.06-7.30 (m, 9H), 7.51 (t, 2H, J=7.6 Hz), 7.57-7.63 (m, 1H), 7.78-7.86 (m, 2H); ¹³C NMR (100 MHz, CDCl₃) δ 31.8, 35.7, 38.9, 43.8, 46.0, 49.1, 54.5, 56.0, 126.2, 127.1, 127.6, 128.4, 128.5, 128.6, 129.3, 129.4, 130.5, 133.5, 136.7, 140.4, 140.5, 145.9, 157.0, 172.1; LC-MS m/z 575.35, 576.38 [M+H]⁺, (C₃₂H₃₆N₄O₄S⁺ Calcd 575.27); t_{R}=3.22 min.

### Example 10

### N-((S)-1-oxo-3-phenyl-1-(((S,E)-5-phenyl-1-(phenylsulfonyl)pent-1-en-3-yl)amino)propan-2-yl)-4-(prop-2-yn-1-yl)piperazine-1-carboxamide (N4-Propargyl-K11777, N4(Propargyl)Pip-Phe-hPhe-VSPh)

Followed the procedure from synthesis of K11777, using TFA salt 5 (1.513 g, 3.640 mmol), DCM (60 mL), acid 16 (1.915 g, 6.072 mmol), DIPEA (8.50 mL, 49 mmol) and T3P (5.80 mL, 9.1 mmol). White solid, 1.553 g, 2.594 mmol, 71% yield. ¹H NMR (400 MHz, CDCl₃) δ 1.59-1.87 (m, 2H), 2.21 (t, 1H, J=2.4 Hz), 2.36-2.57 (m, 6H), 2.95-3.04 (m, 2H), 3.22-3.37 (m, 6H), 4.53-4.66 (m, 2H), 5.25 (d, 1H, J=7.7 Hz), 6.11 (dd, 1H, J, =1.5 Hz, J₂=15.1 Hz), 6.78 (dd, 1H, J, =5.0 Hz, J₂=15.1 Hz), 6.96-7.03 (m, 2H), 7.07-7.23 (m, 9H), 7.46-7.56 (m, 2H), 7.57-7.63 (m, 1H), 7.79-7.87 (m, 2H); ¹³C NMR (100 MHz, CDCl₃) δ 31.8, 35.7, 38.7, 43.7, 46.8, 49.1, 51.2, 55.9, 73.6, 78.2, 126.2, 127.1, 127.6, 128.4, 128.5, 128.6, 129.3 (2C), 130.5, 133.4, 136.7, 140.3, 140.5, 145.8, 156.9, 172.0; LC-MS m/z 599.25, 600.28 [M+H]⁺, (C₃₄H₃₈N₄O₄S⁺ Calcd 599.27); t_{R}=3.68 min.

### Experimental Methods

### Expression and Purification of Main Protease (3CL Protease)

All genes and plasmids utilized for the expression of 3CLpro were prepared by Genscript and any subsequent subcloning of these genes was verified by sequencing. All chemical reagents for protein purification, synthesis, and HPLC were obtained from commercial vendors and used without further purification.

Based on the three-dimensional structure of SARS CoV-2 3CLpro (PDB 6Y84.pdb), we designed expression constructs comprising nucleotides 10055-10972 of ORF1AB from GenBank (protein ID QH062106.1, genome sequence MN988668.1, which encodes 306 amino acids. This gene was E. coli optimized and ligated into the into the BamHI and XhoI restriction sites of the pGEX-6p1 plasmid (Genscript, USA), resulting in the 3CLpro coding sequence being flanked by an N-terminal GST domain followed by a 3CLpro cleavage sequence (SAVLQ*SGF) and an C-terminal sequence containing a modified PreScission protease sequence (SGVTFQ*GP) preceding a His₆ sequence. Upon expression, auto-proteolysis from 3CLpro removed the N-terminal GST tag, yielding the authentic N-terminus (Ser-Gly-Phe). Purification of the processed protein using immobilized nickel affinity chromatography, followed by treatment with HRV 3C protease then generates 3CLpro with authentic N and C termini.

The construct was transformed into BL21-DE3 E. coli cells for protein expression and a single colony of the transformed cells was used to inoculate a culture of LB broth containing 100 ug/mL ampicillin and was incubated at 37° C. overnight. Subsequently, 1 L of LB media containing 100 ug/mL ampicillin was inoculated with the starter culture, and incubated at 37° C. until reaching O.D. 600 of 0.6-0.8, at which time expression was induced by the addition of 1 mM isopropyl β-thiogalactoside (IPTG). The cells were allowed to continue growth at 37° C. for 4-5 h. and were then harvested by centrifugation (6,300 g at 4° C.), and either stored at −80° C. or lysed immediately for purification.

Purification.

Cells were suspended in 12 mM Tris-HCl, 120 mM NaCl, 0.1 mM EDTA, 2 mM DTT, pH: 7.5 (Buffer A). The cell slurry was then lysed using either a French press (25,000 psi) or by sonication. Lysates were centrifuged at 26,000 g to remove cell debris, and clarified lysates were filtered with a 0.45 μm filter. The filtrates were loaded onto a HisTrap HP column (GE Healthcare), and washed with Buffer A. This was directly followed by elution using a linear gradient to 35% buffer B (12 mM Tris-HCl, 120 mM NaCl, 500 mM imidazole, 0.1 mM EDTA, 2 mM DTT, pH: 7.5) over 25 column volumes. The fractions containing pure 3CLpro-PreSision site-His₆ protein, as determined by SDS-PAGE, were pooled, and the protein concentration was determined using the presumed monomeric molecular weight of 33.9 kDa and an extinction coefficient of 32,800 M⁻¹ cm⁻¹. 3CLpro was twice dialyzed against buffer A at 4° C. Proteolysis of the C-terminal He tag of the auto processed translated gene product from the pGEX-6p1 plasmid (3CLpro-HRV 3C protease-Hise) was conducted by incubating 3.5 units of Pierce™ HRV 3C Protease (Thermo Fisher Scientific) per mg of 3CLpro overnight at 4° C. overnight in buffer A. Subsequently, the protein mixture was successively loaded onto a 5 mL GSTrap HP column and a 5-mL HisTrap HP column (GE Healthcare), to remove the GST-fused HRV 3C protease and undigested He tagged protein. The flow through was collected, analyzed by SDS-PAGE, and pure fractions of the tagless 3CLpro were pooled and concentrated (10 kDa molecular weight cutoff filter, GE Healthcare). The protein was deemed to be ≥95% pure by SDS-PAGE, and was stored at −80° C. in 12 mM Tris-HCl, 120 mM NaCl, 0.1 mM EDTA, 2 mM DTT, (pH 7.5) with 50% glycerol (v/v). Analytical gel filtration using a Superdex 200 Increase 10/300 GL column (Buffer A at a flow rate of 0.7 mL/min), indicated that native 3CLpro was the expected homodimer.

### Kinetic Assays of Main Protease (3CLpro)

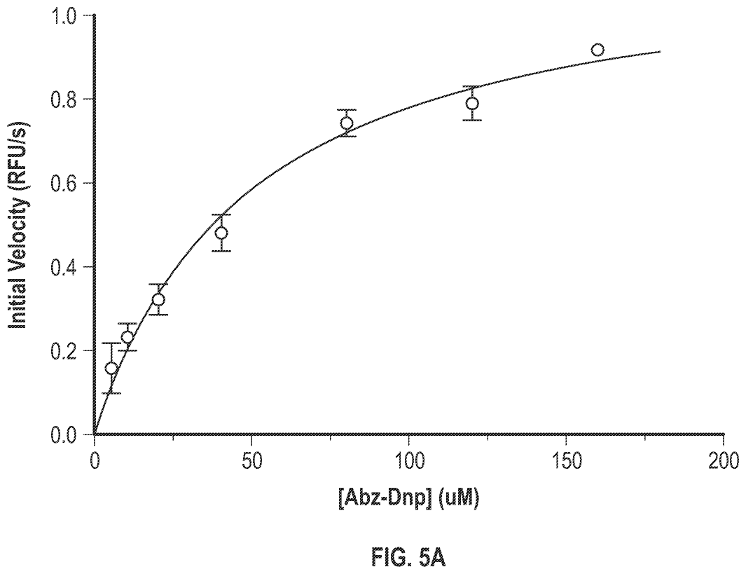

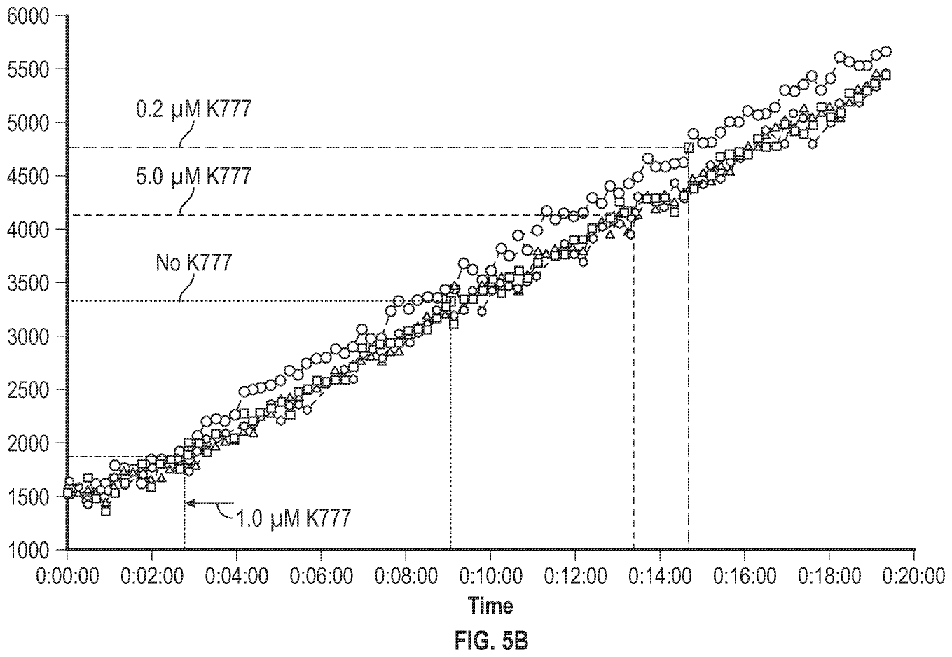

Unless otherwise specified, all assays were conducted at 25° C. in 50-μL reaction mixtures containing 20 mM Tris, 150 mM NaCl, 0.1 mM EDTA, 2 mM DTT, 10% DMSO, and 50 nM 3CL-PR using 96-well plates (Greiner, flat-bottom half volume, clear black plates). Initial rates of the peptidolysis of the FRET-based substrate Abz-Ser-Ala-Val-Leu-Gln*Ser-Gly-Phe-Arg-Lys-(DNP)-NH2was quantified by analyzing the absorbance of 2,4-DNP at 365 nm and using the extinction coefficient of 17,300 M⁻¹ cm⁻¹.²⁶ All enzymatic rates were calculated from standard curves for the fluorophore, permitting the conversion of relative fluorescence units (RFUs) to product concentrations. The enzyme was incubated with varied concentrations of the FRET substrate and monitored until the change in fluorescence was stable. Fluorescence intensity was then replotted vs. substrate concentration, providing where one can divide the RFU/rate by the slope of this line and convert the RFU value to concentration/rate. Initial velocity of this substrate is shown in FIG. 5A, along with the effect of K11777 on 3CLpro enzymatic activity.

### 3CLpro (Main Protease) Kinetic Assay and Evaluation of K777.

Addition of the FRET-based peptide substrate Abz-Ser-Ala-Val-Leu-Gln*Ser-Gly-Phe-Arg-Lys-(DNP)-NH2 at concentrations of 5-160 mM into reaction mixtures containing buffer (pH 7.5, 25° C.) and 40-50 nM purified 3CLpro produced increasing fluorescence over a linear time course for at least 15 min. Plotting of these initial velocities vs. substrate concentration yielded a rectangular hyperbola (FIG. 5A). Fitting of the data to the Michaelis-Menten equation provided the kinetic parameters: Kₘ=66±9 mM, k_{cat}=4.9±0.4 s⁻¹ and k_{cat}/Kₘ=74,000 M⁻¹ s⁻¹, which indicates that this assay is suitable for the analysis of inhibitors of 3CLpro. Reaction mixtures containing 50 mM substrate and 0-5 mM K11777 were treated with 50 nM 3CLpro, and the resulting time courses over 20 min are seen in FIG. 5B. K11777 neither inhibited, or inactivated the 3CLpro-catalyzed proteolytic reaction at micromolar concentrations, indicating that an apparent value of IC₅₀ for K11777 vs. 3CLpro would significantly exceed 10 mM. Accordingly, the selectivity of K11777 for cathepsin L and other clan CA cysteine proteases found in FIG. 3 is greater than 400-fold. These findings indicate that any anti-coronaviral activity exhibited by K11777 cannot be due to inhibition of 3CLpro.

### Kinetic Assays for Cathepsins

For assays of cathepsin B, L, and S, cruzain inhibitors were evaluated in reaction mixtures containing a buffer of sodium acetate (pH 5.5), 1 mM CHAPS, 1 mM Na₂EDTA, and 5 mM DTT at 25° C. The substrate Cbz-Phe-Arg-AMC was dissolved in 100% DMSO, as were all inhibitors, and aliquots of both substrates and inhibitors were added to 0.25 mL reaction mixtures to final concentrations of 10% DMSO (v/v). Michaelis constants for Cbz-Phe-Arg-AMC were determined for all three human cathepsins as cathepsin L (2.9 μM), cathepsin S (60 μM), and cathepsin B (150 μM), and fixed concentrations of Cbz-Phe-Arg-AMC of 1 or 2 Km were used to evaluate inhibitors. Cruzain inhibitors were added at seven concentrations and one fixed concentration of Cbz-Phe-Arg-AMC, and time courses of AMC formation were analyzed as with cruzain.

### Bio-Orthogonal Labeling of Protein Targets of K11777 in Virally-Infected Cells by 4N-Propargyl-K11777.

4N-propargyl-K11777 was added at concentrations of 1-10 □M to Vero E6 (African green monkey kidney cells), with and without infection by SARS CoV-2 viral particles at an multiplicity of infection of 0.1. For control samples, Vero E6 cells were first pre-treated with K11777 (1 μM) for 1 hour, prior to the addition of 4N-propargyl-K11777 (1 μM). Cells growth continued for 4 days, followed by harvest, and ˜ 20 million cells were suspended in 1 mL of lysis buffer (200 mM Tris, 4% CHAPS, 1M NaCl, 8 M Urea, pH=8) and protease inhibitor cocktail (Sigma Aldrich) at a ratio of 50:1 (v/v). Cells were lysed by sonication (ThermoFisher Scientific), with the sample on ice, with a ⅛-inch probe at 20% amplitude for 3-seconds, three times with a minute delay between pulses. The cell debris was pelleted (10,000×g for 10 minutes) followed by removal of the supernatant and determination of protein concentration by BCA assay (ThermoFisher Scientific). Labeling of the K11777-alkyne-treated cells was performed in a stepwise fashion whereby 100 mM TBTA in 1:4 DMSO: n-butanol (v/v) was added to 1 mM CuSO₄ in water, followed by the addition of 20 mM Cy7-azide (Click Chemistry Tools) in DMSO, 24 μg cell lysate, and finally 2.5 mM sodium ascorbate in water. This reaction was incubated at room temperature for 1-3 hours and quenched by the addition ice-cold methanol (200 μL), chloroform (75 μL), and water (150 μL) followed by vortexing and centrifugation at 15,000 RPM at 4° C. for 15 minutes. The top layer of liquid was carefully removed and discarded, and the protein precipitate formed between the two liquid phases was undisturbed. An additional 1 mL of ice-cold methanol was added into the tube, followed by vortexing, and centrifugation at 15,000 rpm at 4° C. for 20 minutes. The supernatant was discarded, and this step was repeated. Following removal of the supernatant the protein pellet was allowed to air-dry at room temperature. To the dried pellet 4% SDS buffer (4% SDS (w/v), 50 mM TEA, 150 mM NaCl, pH 7.4) was added and the pellet was sonicated to achieve a clear solution. Then, 4× loading dye (240 mM Tris-HCl, 8% SDS (w/v), 0.004% bromophenol blue (to avoid fluorescence from the dye), 5 mM DTT, and MilliQ water was added to a total volume of 24 μL. Samples were heated to 95° C. for 5 minutes followed by a brief centrifugation at 5,000 RPM for 1 min. The protein-standards ladder (Bio-Rad, Precision Plus Protein™ Dual Color Standards) was diluted 10,000 fold in 4% SDS buffer, loading dye, and water and loading dye 4× loading dye and water to avoid strong fluorescence from the ladder during imaging. Samples were analyzed on a 12% Bis-Tris Nu-PAGE gel (Invitrogen). To each well, 20 μg of labeled lysate was loaded and the gel was run at 60 V for 30 minutes followed by 120 V for ˜90 minutes. The gels were destained (10% acetic acid, 40% methanol, 50% water) for 1 hour-overnight in the dark followed by imaging the gel on a ChemiDoc imager (Bio-Rad) using the Cy7 blot setting. Densitometry of in gel fluorescence was conducted using ImageJ. The gels were also analyzed by Coomassie Brilliant Blue R-250 staining (Bio-Rad).

### Azide Enrichment and Proteomic Analysis of Vero E6 K11777-Alkyne Treated Cells

Cell lysates of Vero E6 cells were prepared as described above. Enrichment of alkyne-modified proteins was conducted using the Click-&-Go Protein Enrichment Kit (Click Chemistry Tools) and the protocols thereof. Proteolysis of the resin-bound, enriched proteins, which had been reduced with DTT and alkylated with iodoacetamide, was conducted by addition of 1 μg of Trypsin Gold (Promega) at 37° C. overnight with gentle agitation. The digested peptides were then eluted from the column and subjected to desalting using Pierce™ C18 Tips, 100 μL bed (Thermo Fisher Scientific). The desalted peptides were concentrated to dryness via speed vacuum centrifugation, and suspended in 25 mM ammonium bicarbonate. LC-MS/MS analysis was performed using an UltiMate 3000 HPLC system (Thermo Scientific) coupled to a Thermo Scientific Orbitrap Fusion™ Tribrid™ mass spectrometer. 1 μL of each sample was injected onto a PepMap100 C18 5 μm trap cartridge (0.3×5 mm) followed by an Acclaim PepMap C18 column (0.075 mm×150 mm, particle size 3 μm, pore size 100 Å) at a flow rate of 30 μL/min. Peptides were eluted at a flow rate of 0.200 μL/min, using 98% water/2% acetonitrile with 0.1% formic acid (A) and 2% water/98% acetonitrile (v/v) with 0.1% formic acid (v/v) (B) as mobile phase. The gradient used was as follows: equilibration at 2% B for 5 minutes, increasing to 45% at 37 minutes, 90% B at 40 to 46 minutes, and decreasing to 2% B at 47 minutes, followed by re-equilibration at 2% B until the end of the run (60 minutes).

Following LC separation, samples were introduced into the mass spectrometer by nanoelectrospray ionization at a spray voltage of 2450 V, with the ion transfer tube temperature set to 275° C. Data were acquired in top-speed mode, with the cycle duration set to 3 seconds. Full scan data were acquired on an Obitrap apparatus set in the 400-1600 m/z range at a resolution of 120,000 at m/z 200. MS/MS data were acquired in the ion trap in rapid scan mode, using an isolation window of 1.6 m/z with HCD at a fixed normalized collision energy of 28%. Dynamic exclusion was set to 60 seconds.

Data were processed using MaxQuant 1.6.14. Label-free quantification was enabled at the default parameters. The green monkey (Chlorocebus sabaeus) proteome (UniProt ID UP000029965; 19,229 protein sequences) was used as the target database. MaxQuant result files were analyzed using Perseus 1.6.14. Reverse hits, potential contaminant hits, hits only identified by site and hits for which only one peptide was detected were removed. Only proteins which were not detected in the control were considered. Intensity values were converted to logarithms. Finally, proteins which were detected in at least two test samples were clustered based on highest to lowest intensity in the 5 μM and 10 μM treated columns at default parameters (column clustering was disabled) and plotted as a heat map.

### Western Blotting

Cell lysates were prepared as stated above. The samples were diluted in 4× loading dye (240 mM Tris-HCl, 8% SDS(w/v), 0.02% bromophenol blue, 5 mM DTT), and MilliQ water, and heated to 95° C. for 5 minutes followed by a brief centrifugation at 5,000 rpm for 1 min. 20 mg of protein for each total lysate was submitted to SDS-PAGE (12% Bis-Tris Nu-PAGE gel, Invitrogen). The protein(s) were transferred onto an activated PDVF membrane (GE Healthcare Life Sciences) using a semi-dry blot transfer (Bio-Rad) at 25V for 20 minutes using transfer buffer (25 mM Tris-HCl, 192 mM glycine, 20% methanol, pH 7.6). The membrane was then submerged into TBST buffer (20 mM Tris-HCl, 150 mM NaCl, 0.1% Tween 20. pH 7.5) containing 5% (w/v) dried milk and allowed to incubate at room temperature for one hour under constant agitation. Anti-actin antibody (Sigma Aldrich) or anti-Flag (Cell Signaling Technologies) was added at a 1:100,000 or 1:25,000 dilution respectively, and allowed to incubate overnight at 4° C. under constant agitation. The membrane was subsequently washed 5× with TBST and then probed with the goat-anti rabbit HRP conjugate antibody (Bio-Rad) was diluted 1:3000 in TBST containing 5% wt/vol milk for one hour at room temperature. The membrane was washed five times with TBST, followed by addition of the ECL substrate (Bio-Rad), and imaged on a ChemiDoc imager (Bio-Rad) using the chemiluminescence blot setting. Stripping and reprobing of blots was conducted by washing the blot five times with TBS buffer (20 mM Tris-HCl, 150 mM NaCl, pH 7.5) followed by incubation with stripping buffer for 15 minutes (Restore Stripping buffer, ThermoFisher Scientific). The blot was then washed five times with TBS buffer, blocked with 5% milk (w/v) in TBST for one hour, and probed with the appropriate antibody(s). The protein content of the membrane was visualized by Ponceau S staining (Sigma Aldrich). Densitometry analysis of Western blots was performed using ImageJ.

### Example 11

### Proteolysis of SARS CoV-1 and -2 Spike Protein by Trypsin, Cathepsin B, and L

Recombinant SARS CoV-2 and SARS CoV-1 spike protein(s) were purchased from commercial vendors, and used without additional purification. For SARS CoV-2 S we used protein expressed in Sf9 insect cells containing the S1, S2, and extracellular (ECD) domains containing a C-terminal Flag-hexahistidine tag, found to be >90% pure (Genscript). We purchased SARS-CoV-2 trimeric spike protein containing a C-terminal hexahistidine tag expressed in HEK293 cells, which contained the mutations (R683A, R685A) which are the sites of furin-catalyzed proteolysis (found to be 95% pure by SDS-Page (Acro Biosystem)). For the SARS CoV-1 S protein we used protein expressed from a baculoviral vector in Sf9 cells, containing the S1, S2, and ECD domain and a C-terminal hexahistidine tag from Sino Biological. Subsequently, for all experiments, 2.5 μg of the above proteins were incubated with trypsin (25 nM) in 50 mM HEPES, 1 mM Na-EDTA, pH: 7.5 or with cathepsin B (250 nM), or cathepsin L (2-250 nM) in 50 mM sodium acetate, 1 mM Na-EDTA, 1 mM CHAPS, and 1 mM DTT, pH: 5.5. These studies were performed in both the presence and absence of inhibitors for one hour at room temperature in which the inhibitors (100 μM leupeptin and 2.5-5 μM K777, final) were dissolved in DMSO at a final concentration of 10% (v/v). Proteolysis reaction mixtures of SARS-CoV-2 or SARS-CoV-1 S proteins with each protease in the absence of inhibitors contained no DMSO. Following 1 hour of cleavage, all reactions were quenched by addition of 4×SDS-PAGE loading dye containing 5 mM DTT, and heated at 95° C. for 10 minutes. The samples were then loaded onto a 4-12% Bis-Tris Nu-PAGE gel (Invitrogen) using PAGE Ruler loading dye (Invitrogen) to estimate the molecular weights, and electrophoresed at 80 V for 25 minutes followed by 120 V for ˜120 mins. The gels were then either stained with Coomassie Blue, or transferred to a PDVF membrane for Western blotting as described above. Imaging of the gels was conducted on a ChemiDoc imager (Bio-Rad). Quantitation of gels was conducted using ImageJ.

### Example 12

### Evaluation of K11777 in SARS-CoV-2 Infection Assay

K11777 was dissolved in 100% DMSO as 10 mM stock solutions and diluted in culture media to final concentrations of DMSO of 1% (v/v). Vero E6, A549 expressing human ACE2 receptor (A549/ACE2) or Calu-3 (2B4 clone) grown in 96-well microtiter plates will be pre-treated with serially 2-fold dilution of drug for two hours before infection with 100 (Vero E6 cells) or 500 (A549/ACE2 and 2B4 cells) infectious SARS-CoV-2 (US_WA-1 isolate) particles in 100 μl EMEM supplemented with 2% FBS. Cells pre-treated with parallelly diluted dimethyl sulfoxide (DMSO) with or without virus will be included as positive and negative controls, respectively, as for evaluating the potential of DMSO and/or drugs related cytotoxicity. After cultivation at 37° C. for 3 days (Vero E6) or 4 days (A549/ACE2), individual wells will be observed under the microcopy for the status of virus-induced formation of CPE. The efficacy of individual drugs will be calculated and expressed as the lowest concentration capable of completely preventing virus-induced CPE in 50% (EC₅₀) or in 100% (EC₁₀₀) of the wells. All compounds will be prepared in 100% DMSO as 10 mM stock solutions and diluted in culture media. Since 2B4 cells did not show obvious CPEs until 9 dpi, at day 4 p.i., supernatants from infected cells were harvested and tittered in Vero E6 cells grown in 96-well microtiter plates using the standard infectivity assay and the titers of infectious virus were expressed as log₁₀ TCID₅₀/mL. FIG. 4 shows the effects of K777 in Vero E6 and A549/ACE2 cells, infected with SARS CoV-2. Measurement of the cytopathic effect (CPE) indicates the extent to which SAR-CoV-2 has killed the host cells, and values of EC₅₀ indicate the concentrations of K777 that reduce the CPE by 50%.

### Example 12

The methods described herein (e.g., method of treating a coronavirus infection in a subject in need thereof) are general across various coronaviruses including MERS and SARS-CoV coronaviruses. As shown in Table 1 below, at least the compound referenced herein as K777 is active against SARS CoV-2, SARS CoV-1, and MERS. These data are summarized from the Mellott, et al. ACS Chem. Biol. 2021, 16, 642-650, article and accompanying Supplementary Information (FIG. B1 on page 12), the article and the Supplementary Information being incorporated by reference as if fully set forth herein in their entirety.

TABLE 1

| Coronavirus | Host Cell | EC₅₀ (mM) |
|---|---|---|
| SARS CoV-2 | Vero E6 | 0.6 |
| SARS CoV-2 | A549/ACE2 | <0.078 |
| SARS CoV-1 | Vero E6 | 2.5 |
| SARS CoV-1 | A549/ACE2 | 0.08 |
| MERS | Vero E6 | 0.6 |
| MERS | A549/ACE2 | 0.078 |

The disclosure provides for the following example embodiments, the numbering of which is not to be construed as designating levels of importance:

Embodiment 1 relates to a pharmaceutical composition comprising a compound having Formula I:

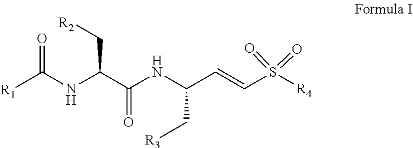

or a pharmaceutically acceptable salt or derivatives thereof,
wherein
R₁-R₄ are independently a substituted or unsubstituted alkyl, alkenyl, alkynyl, aryl, heteroaryl, or heterocycloalkyl; and one or more pharmaceutically acceptable carriers; wherein the compound is present in an effective amount to inhibit a non-viral cysteine protease.

Embodiment 2 relates to the composition of Embodiment 1, wherein R₁ is a heteroaryl.

Embodiment 3 relates to the composition of Embodiment 1, wherein R₁ is heterocycloalkyl.

Embodiment 4 relates to the composition of Embodiment 1, wherein R₁ is N-methyl-piperazinyl or a N-propynyl-piperazinyl.

Embodiment 5 relates to the composition of Embodiment 1, wherein R₂-R₄ are independently phenyl, substituted phenyl, benzyl, substituted benzyl, 4-pyridine, or 3-amino-propyl.

Embodiment 6 relates to the composition of Embodiment 1, wherein Rr R4 are substituted or unsubstituted phenyl.

Embodiment 7 relates to the composition of Embodiment, wherein R₂ and R₄ are phenyl.

Embodiment 8 relates to the composition of Embodiment 1, wherein R₃ is benzyl.

Embodiment 9 relates to the composition of Embodiment 1, wherein the compound of Formula I is:

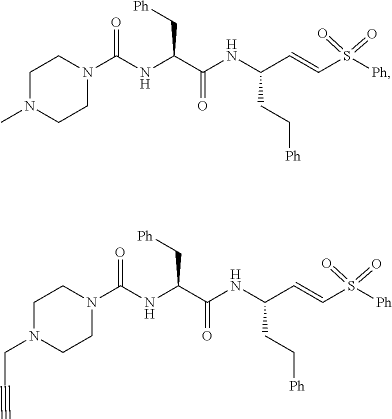 [SMILES: CN1CCN(C(=O)N[C@@H](Cc2ccccc2)C(=O)N[C@H](/C=C/S(=O)(=O)c2ccccc2)CCc2ccccc2)CC1 ; C#CCN1CCN(C(=O)N[C@@H](Cc2ccccc2)C(=O)N[C@H](/C=C/S(=O)(=O)c2ccccc2)CCc2ccccc2)CC1]

pharmaceutically acceptable salts or derivatives thereof.

Embodiment 10 relates to the composition of Embodiment 1, wherein the non-viral cysteine protease is a mammalian cysteine protease.

Embodiment 11 relates to the composition of Embodiment 1, wherein the non-viral cysteine protease is selected from cruzain, cruzipain, cathepsin L, cathepsin B, cathepsin K, cathepsin S, cathepsin V, or other cysteine proteases.

Embodiment 12 relates to the composition of Embodiment 11, wherein the mammalian cysteine protease is cathepsin L.

Embodiment 13 relates to the composition of Embodiment 12, wherein the compound is a cathepsin L inhibitor.

Embodiment 14 relates to a method of inhibiting a non-viral cysteine protease comprising administering a therapeutically effective amount of the compound of Embodiment 1 to a subject.

Embodiment 15 relates to the method Embodiment 14, wherein the non-viral cysteine protease is a mammalian cysteine protease.

Embodiment 16 relates to the method of Embodiment 15, wherein the mammalian cysteine protease is cathepsin L.

Embodiment 17 relates to a method of treating a coronavirus infection in a subject in need thereof, the method comprising administering a therapeutically effective amount of the compound of Embodiment 1 to a subject.

Embodiment 18 relates to the method of Embodiment 17, wherein the coronavirus infection is caused by SARS-CoV-2.

Embodiment 19 relates to a method of identifying a protein target of a compound having Formula I,

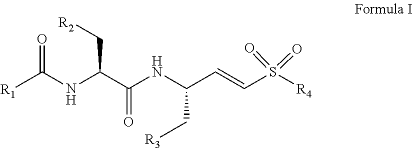

or a pharmaceutically acceptable salt or derivatives thereof,
wherein
R₁-R₄ are independently a substituted or unsubstituted alkyl, alkenyl, alkynyl, aryl, heteroaryl, or heterocycloalkyl;
the method comprising:
contacting the protein target with a click target defined by Formula I, wherein at least one of R₁, R₂, R₃, and/or R₄ comprises a click motif;
contacting the click target with a click complement, thereby labeling the protein target;
and detecting the labeled protein target.

Embodiment 20 relates to a method of identifying a protein target of a compound having Formula I in mammalian cells or tissues infected or not-infected with at least one coronavirus,

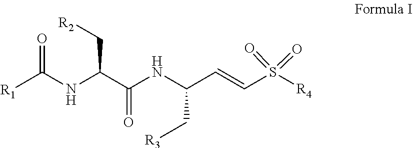

or a pharmaceutically acceptable salt or derivatives thereof,
wherein
R₁-R₄ are independently a substituted or unsubstituted alkyl, alkenyl, alkynyl, aryl, heteroaryl, or heterocycloalkyl;
the method comprising:
labeling a protein target in mammalian cells or tissues infected or not-infected with coronaviruses with a click target defined by Formula I, wherein at least one of R₁, R₂, R₃, and/or R₄ comprises a click motif;
contacting the click target with a click complement, thereby labeling the protein target; and
detecting the labeled protein target.

Embodiment 21 relates to the method of Embodiment 18 or Embodiment 19, wherein the click target is defined by Formula II:

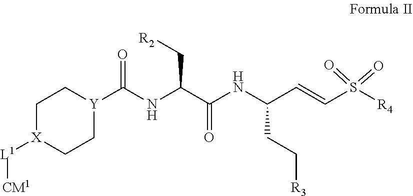

wherein
X and Y are independently N, O, or S; R₂-R₄ are independently a substituted or unsubstituted alkyl, alkenyl, alkynyl, aryl, heteroaryl, or heteroalkyl; and
L¹ is absent, or represents a linking group; and CM¹ represents a first click motif.

Embodiment 22 relates to the method of Embodiment 18 or Embodiment 19, wherein the click motif is an alkyne.

Embodiment 23 relates to the method of Embodiment 18 or Embodiment 19, wherein the click complement comprises a second click motif.

Embodiment 24 relates to the method of Embodiment 23, wherein the second click motif is an azide.

Embodiment 25 relates to the method of Embodiment 18 or Embodiment 19, wherein the click target is:

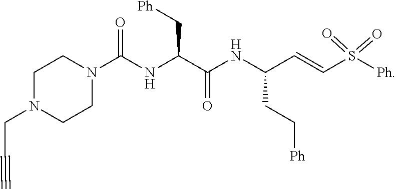 [SMILES: C#CCN1CCN(C(=O)N[C@@H](Cc2ccccc2)C(=O)N[C@H](/C=C/S(=O)(=O)c2ccccc2)CCc2ccccc2)CC1]

Embodiment 26 relates to the method of Embodiment 18 or Embodiment 19, wherein the protein target is cruzain, cruzipain, cathepsin B, cathepsin L, cathepsin S, or other cysteine proteases.

Embodiment 27 relates to a method of treating a coronavirus disease, the method comprising administering a composition of Embodiment 1 to a subject.

Embodiment 28 relates to the method of Embodiment 27, wherein the coronavirus disease is COVID-19.

Embodiment 29 relates to a method of preventing or delaying the onset or progression of a coronavirus disease, the method comprising administering a composition of Embodiment 1 to a subject.

Embodiment 30 relates to the method of Embodiment 29, wherein the coronavirus disease is COVID-19.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A pharmaceutical composition comprising a compound having Formula I: 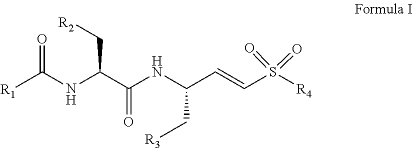
or a pharmaceutically acceptable salt or derivatives thereof,
wherein
R₁-R₄ are independently a substituted or unsubstituted alkyl, alkenyl, alkynyl, aryl, heteroaryl, or heterocycloalkyl; and one or more pharmaceutically acceptable carriers;
wherein the compound is present in an effective amount to inhibit a non-viral cysteine protease.

2. The composition of claim 1, wherein R₁ is a heteroaryl.

3. The composition of claim 1, wherein R₁ is heterocycloalkyl.

4. (canceled)

5. The composition of claim 1, wherein R₂-R₄ are independently phenyl, substituted phenyl, benzyl, substituted benzyl, 4-pyridine, or 3-amino-propyl.

6. (canceled)

7. (canceled)

8. (canceled)

9. The composition of claim 1, wherein the compound of Formula I is: 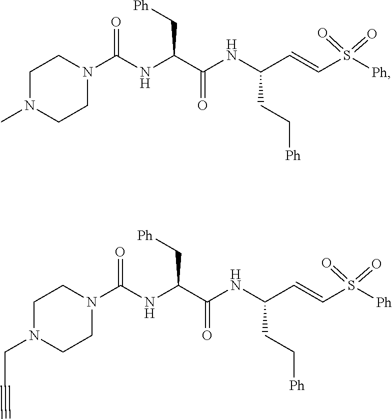 [SMILES: CN1CCN(C(=O)N[C@@H](Cc2ccccc2)C(=O)N[C@H](/C=C/S(=O)(=O)c2ccccc2)CCc2ccccc2)CC1 ; C#CCN1CCN(C(=O)N[C@@H](Cc2ccccc2)C(=O)N[C@H](/C=C/S(=O)(=O)c2ccccc2)CCc2ccccc2)CC1]
or pharmaceutically acceptable salts or derivatives thereof.

10. (canceled)

11. (canceled)

12. (canceled)

13. (canceled)

14. A method of inhibiting a non-viral cysteine protease comprising administering a therapeutically effective amount of the composition of claim 1 to a subject.

15. The method of claim 14, wherein the non-viral cysteine protease is a mammalian cysteine protease.

16. The method of claim 15, wherein the mammalian cysteine protease is cathepsin L.

17. A method of treating a coronavirus infection in a subject in need thereof, the method comprising administering a therapeutically effective amount of the composition of claim 1 to a subject.

18. The method of claim 17, wherein the coronavirus infection is caused by SARS-CoV-2.

19. A method of identifying a protein target of a compound having Formula I, 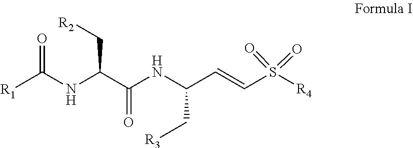
or a pharmaceutically acceptable salt or derivatives thereof,
wherein
R₁-R₄ are independently a substituted or unsubstituted alkyl, alkenyl, alkynyl, aryl, heteroaryl, or heterocycloalkyl;
the method comprising:
contacting the protein target with a click target defined by Formula I, wherein at least one of R₁, R₂, R₃, and/or R₄ comprises a click motif;
contacting the click target with a click complement, thereby labeling the protein target; and
detecting the labeled protein target.

20. A method of identifying a protein target of a compound having Formula I in mammalian cells or tissues infected or not-infected with at least one coronavirus, 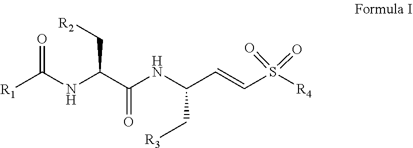
or a pharmaceutically acceptable salt or derivatives thereof,
wherein
R₁-R₄ are independently a substituted or unsubstituted alkyl, alkenyl, alkynyl, aryl, heteroaryl, or heterocycloalkyl
the method comprising:
labeling a protein target in mammalian cells or tissues infected or not-infected with coronaviruses with a click target defined by Formula I, wherein at least one of R₁, R₂, R₃, and/or R₄ comprises a click motif;
contacting the click target with a click complement, thereby labeling the protein target; and
detecting the labeled protein target.

21. The method of claim 18, wherein the click target is defined by Formula II: 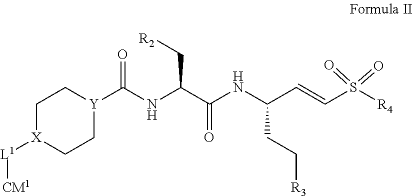
wherein
X and Y are independently N, O, or S; R₂-R₄ are independently a substituted or unsubstituted alkyl, alkenyl, alkynyl, aryl, heteroaryl, or heteroalkyl; and
L¹ is absent, or represents a linking group; and CM¹ represents a first click motif.

22. The method of claim 19, wherein the click motif is an alkyne.

23. The method of claim 19, wherein the click complement comprises a second click motif.

24. The method of claim 23, wherein the second click motif is an azide.

25. The method of claim 19, wherein the click target is:

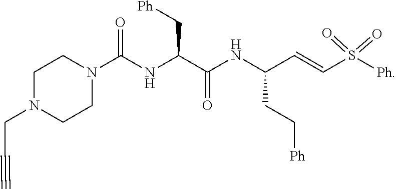 [SMILES: C#CCN1CCN(C(=O)N[C@@H](Cc2ccccc2)C(=O)N[C@H](/C=C/S(=O)(=O)c2ccccc2)CCc2ccccc2)CC1]

26. The method of claim 19, wherein the protein target is cruzain, cruzipain, cathepsin B, cathepsin L, cathepsin S, or other cysteine proteases.

27. A method of treating, preventing or delaying the onset or progression of a coronavirus disease, the method comprising administering a composition of claim 1 to a subject in need thereof.

28. The method of claim 27, wherein the coronavirus disease is COVID-19.

29. (canceled)

30. (canceled)
